SEQUENCE LISTING (44 sequences)

SEQ ID NO: 1 - DNA, 22 bp, Artificial sequence
  Synthetic oligonucleotide
gctgcagatcaagccagagagc

SEQ ID NO: 2 - DNA, 21 bp, Artificial sequence
  Synthetic oligonucleotide
attgatttctgcatgtgctgc

SEQ ID NO: 3 - DNA, 20 bp, Artificial sequence
  Synthetic oligonucleotide
tgactacctaccctgttcag

SEQ ID NO: 4 - DNA, 20 bp, Artificial sequence
  Synthetic oligonucleotide
ttcactggatgtgagctctc

SEQ ID NO: 5 - DNA, 20 bp, Artificial sequence
  Synthetic oligonucleotide
acggcacgtggaaagtagac

SEQ ID NO: 6 - DNA, 20 bp, Artificial sequence
  Synthetic oligonucleotide
agaaacctctcggacagcaa

SEQ ID NO: 7 - DNA, 20 bp, Artificial sequence
  Synthetic oligonucleotide
ggacaggacttcacctggtc

SEQ ID NO: 8 - DNA, 20 bp, Artificial sequence
  Synthetic oligonucleotide
caagccagcctcttgcttac

SEQ ID NO: 9 - DNA, 20 bp, Artificial sequence
  Synthetic oligonucleotide
atgctggagaccaaatggag

SEQ ID NO: 10 - DNA, 20 bp, Artificial sequence
  Synthetic oligonucleotide
cctcatactgggcacgaact

SEQ ID NO: 11 - DNA, 20 bp, Artificial sequence
  Synthetic oligonucleotide
ccgaagtcagacggaaagag

SEQ ID NO: 12 - DNA, 20 bp, Artificial sequence
  Synthetic oligonucleotide
tgctggaggtgcagatgtag

SEQ ID NO: 13 - DNA, 20 bp, Artificial sequence
  Synthetic oligonucleotide
tgttggtgcacttcctaccc

SEQ ID NO: 14 - DNA, 20 bp, Artificial sequence
  Synthetic oligonucleotide
tagagggagcaaatgctggt

SEQ ID NO: 15 - DNA, 20 bp, Artificial sequence
  Synthetic oligonucleotide
tgcaacctcctcgaacgcgg

SEQ ID NO: 16 - DNA, 20 bp, Artificial sequence
  Synthetic oligonucleotide
ccaggatctgctccagagac

SEQ ID NO: 17 - DNA, 20 bp, Artificial sequence
  Synthetic oligonucleotide
ctcagcgaggagaaatggtc

SEQ ID NO: 18 - DNA, 20 bp, Artificial sequence
  Synthetic oligonucleotide
ctcagcgaggagaaatggtc

SEQ ID NO: 19 - DNA, 20 bp, Artificial sequence
  Synthetic oligonucleotide
tggtccctgaggacaagttc

SEQ ID NO: 20 - DNA, 20 bp, Artificial sequence
  Synthetic oligonucleotide
tgaattccgtttccaagagg

SEQ ID NO: 21 - DNA, 20 bp, Artificial sequence
  Synthetic oligonucleotide
atgacatcaagaaggtggtg

SEQ ID NO: 22 - DNA, 20 bp, Artificial sequence
  Synthetic oligonucleotide
cataccaggaaatgagcttg

SEQ ID NO: 23 - DNA, 20 bp, Artificial sequence
  Synthetic oligonucleotide
cgcagggacctgtacttctc

SEQ ID NO: 24 - DNA, 20 bp, Artificial sequence
  Synthetic oligonucleotide
tcaggagaaaggggtacgtg

SEQ ID NO: 25 - DNA, 20 bp, Artificial sequence
  Synthetic oligonucleotide
agcaccgccataaactcaac

SEQ ID NO: 26 - DNA, 20 bp, Artificial sequence
  Synthetic oligonucleotide
atctggttgcatccttggag

SEQ ID NO: 27 - DNA, 20 bp, Artificial sequence
  Synthetic oligonucleotide
aaagaatcaagcgcttttcg

SEQ ID NO: 28 - DNA, 20 bp, Artificial sequence
  Synthetic oligonucleotide
tccattgtctgtccgtttga

SEQ ID NO: 29 - DNA, 20 bp, Artificial sequence
  Synthetic oligonucleotide
taatcgaaaaggcccacaac

SEQ ID NO: 30 - DNA, 20 bp, Artificial sequence
  Synthetic oligonucleotide
atgttgctttccttgccaac

SEQ ID NO: 31 - DNA, 20 bp, Artificial sequence
  Synthetic oligonucleotide
gacatggacagcatcattgc

SEQ ID NO: 32 - DNA, 20 bp, Artificial sequence
  Synthetic oligonucleotide
ggctctgcagctcctcatac

SEQ ID NO: 33 - DNA, 20 bp, Artificial sequence
  Synthetic oligonucleotide
agcttggtggtggatgaaac

SEQ ID NO: 34 - DNA, 20 bp, Artificial sequence
  Synthetic oligonucleotide
tcttgcttcatcgtttgcag

SEQ ID NO: 35 - DNA, 20 bp, Artificial sequence
  Synthetic oligonucleotide
gggaacgaggcttcttctac

SEQ ID NO: 36 - DNA, 18 bp, Artificial sequence
  Synthetic oligonucleotide
cacaatgccacgcttctg

SEQ ID NO: 37 - DNA, 20 bp, Artificial sequence
  Synthetic oligonucleotide
cttcgcagggaaagagtgtc

SEQ ID NO: 38 - DNA, 20 bp, Artificial sequence
  Synthetic oligonucleotide
gactgctcagcattgttgga

SEQ ID NO: 39 - DNA, 20 bp, Artificial sequence
  Synthetic oligonucleotide
ctcagcgaggagaaatggtc

SEQ ID NO: 40 - DNA, 20 bp, Artificial sequence
  Synthetic oligonucleotide
ctcagcgaggagaaatggtc

SEQ ID NO: 41 - DNA, 20 bp, Artificial sequence
  Synthetic oligonucleotide
gaacgggattcagaccatgt

SEQ ID NO: 42 - DNA, 20 bp, Artificial sequence
  Synthetic oligonucleotide
aacctggatgcacttcttgg

SEQ ID NO: 43 - DNA, 20 bp, Artificial sequence
  Synthetic oligonucleotide
tcacctgcttcttccgagat

SEQ ID NO: 44 - DNA, 20 bp, Artificial sequence
  Synthetic oligonucleotide
gaggagaaccagcactccag

(19) United States
(12) Patent Application Publication
(10) Pub. No.: US 2011/0150845 A1
(43) Pub. Date: Jun. 23, 2011

(54) METHODS AND COMPOSITIONS FOR MODULATING IMMUNOLOGICAL TOLERANCE

(75) Inventors: Biju Parekkadan, Cambridge, MA (US); Martin Leon Yarmush, Newton, MA (US); Shannon J. Turley, West Roxbury, MA (US)

(73) Assignees: MASSACHUSETTS INSTITUTE OF TECHNOLOGY, Cambridge, MA (US); THE GENERAL HOSPITAL CORPORATION D/B/A MASSACHUSETTS GENERAL HOSPITAL, Boston, MA (US); DANA-FARBER CANCER INSTITUTE, INC., Boston, MA (US)

(21) Appl. No.: 12/990,331

(22) PCT Filed: May 1, 2009
(86) PCT No.: PCT/US2009/002700

§ 371 Date: Jan. 5, 2011

Related U.S. Application Data
(60) Provisional application No. 61/126,310, filed on May 2, 2008.

(51) Int. Cl. A61K 35/12; C12N 5/0775; C40B 40/02; C12Q 1/02; A61P 37/00

(52) U.S. Cl. 424/93.7; 435/325; 506/14; 424/520; 435/29

(57) ABSTRACT

The invention provides compositions and methods for modulating immune responses using mesenchymal stem cells. The invention further provides methods for inducing tolerance to self antigens using mesenchymal stem cells.

### RELATED APPLICATIONS

This application claims priority under 35 U.S.C. §119 from U.S. provisional application Ser. No. 61/126,310, filed May 2, 2008, the entire contents of which are incorporated by reference herein.

### BACKGROUND OF THE INVENTION

One of the central tenets of immunology states that the immune system must respond appropriately to antigens. The immune system requires a number of checks and balances to respond to antigens appropriately during health and disease. For protein antigens, a T cell response is required and this necessitates a need for T cells to discriminate between self and non-self protein antigens to thereby limit self-reactivity and autoimmune disease. This issue of tolerance first occurs during the development of T cells in a process known as negative selection and this tolerance education is further reinforced in the periphery.

There are resident cell populations in lymphoid organs whose function is to educate T cells to self-antigens. These cell populations rely on a unique ability to promiscuously express self peptide antigens synthesized within the cell itself. In other words, these cells endogenously make peripheral tissue antigens (pTAs), which were typically thought to be “tissue-specific”, in order to induce tolerize T cells to self proteins. pTA expression is regulated by the “master” transcription factor, AIRE. The presentation of self peptides centrally by AIRE+ cells was recently found to be involved in the generation of suppressor T cells, also known as regulatory T cells (Aschenbrenner, K. et al., Nat Immunol 8, 351-8 (2007)). Regulatory T cells promote peripheral tolerance of self-reactive lymphocytes that have evaded thymic selection (Fontenot, J. D. et al., Nat Immunol 4, 330-6 (2003)).

### SUMMARY OF THE INVENTION

The invention is based in part on the novel and unexpected finding that mesenchymal stem cells are able to induce tolerance to self antigens. The invention therefore provides compositions and methods of use thereof relating to mesenchymal stem cells.

Thus, in one aspect the invention provides a method for preparing an isolated mesenchymal stem cell population having a defined antigen expression profile comprising determining an antigen expression profile in an isolated mesenchymal stem cell population, and physically separating the isolated mesenchymal stem cell population based on antigen expression to generate one or more isolated mesenchymal stem cell populations having defined antigen expression profile.

In one embodiment, the antigen expression profile is a peripheral tissue antigen expression profile. In one embodiment, the antigen expression profile is an antigen expression profile for a single antigen. In another embodiment, the antigen expression profile is an antigen expression profile for multiple antigens. In one embodiment, the antigen expression profile is protein expression profile. In one embodiment, the isolated mesenchymal stem cell population is physically separated based on type of antigen expression. In another embodiment, the isolated mesenchymal stem cell population is physically separated based on type and level of antigen expression.

In one embodiment, the isolated mesenchymal stem cell population is a bone marrow mesenchymal stem cell population.

In another aspect, the invention provides a method for preparing a mesenchymal stem cell having a defined antigen profile comprising expressing an exogenous nucleic acid in a mesenchymal stem cell, wherein the exogenous nucleic acid comprises a coding sequence for a peripheral tissue antigen.

In one embodiment, the mesenchymal stem cell is a bone marrow mesenchymal stem cell population.

In another aspect, the invention provides an isolated mesenchymal stem cell population having a defined antigen expression profile, and a composition comprising such an isolated population.

In another aspect, the invention provides an isolated mesenchymal stem cell population prepared according to any of the foregoing methods.

In still another aspect, the invention provides a cell bank comprising one or a plurality of isolated mesenchymal stem cell populations having defined antigen expression profiles, including those prepared by the foregoing methods. The bank may further comprise one or more samples of conditioned media from such cell populations, and/or one or more samples of cellular lysates of such cell populations. The conditioned media or lysates may be fractionated, for example, into a lipid containing fraction (including for example vesicles) and a non-lipid containing fraction. In one embodiment, the cell populations are cryopreserved. In this and other embodiments, the lysates and/or conditioned media samples, and/or fractions of either may be cryopreserved or lyophilized.

In still another aspect, the invention provides a method for treating a subject having or at risk of developing an autoimmune disease, or other condition that would benefit from immune tolerance induction, comprising administering to a subject in need thereof an isolated mesenchymal stem cell population having a defined antigen expression profile in an effective amount to treat the subject. The effective amount may be the amount that reduces to or eliminates symptoms of the disease or condition. The cell population may be prepared according to any of the foregoing methods, but it is not so limited.

In one embodiment, the autoimmune disease is an inflammatory bowel disease (IBD). In one embodiment, the isolated mesenchymal stem cell population expresses a peripheral tissue antigen.

In another aspect, the invention provides a method for modulating an immune response comprising administering to a subject in need thereof an isolated mesenchymal stem cell population having a defined antigen expression profile in an effective amount to modulate the immune response and in some instances to treat the subject. The cell population may be prepared according to any of the foregoing methods, but it is not so limited. The method may additionally or alternatively comprise administering a conditioned media or a lysate from such mesenchymal stem cells, or a fraction thereof (e.g., a lipid containing lysate or conditioned media fraction) to the subject.

In one embodiment, the immune response is an autoimmune response. In another embodiment, the immune response is a graft-versus-host immune response. In one embodiment, the immune response is down-regulated or redirected as a result of mesenchymal stem cell (or conditioned media or lysate) administration.

In another aspect, the invention provides a method for treating a subject having or at risk of developing an autoimmune disease comprising administering to a subject in need thereof a mesenchymal stem cell lysate lipophilic fraction in an effective amount to treat the subject. In one embodiment, the lysate fraction is derived from an isolated mesenchymal stem cell population having a defined antigen expression profile and is enriched for lipids.

In a related aspect, the invention provides a method for treating a subject having or at risk of developing an autoimmune disease comprising administering to a subject in need thereof a mesenchymal stem cell conditioned media lipophilic fraction in an effective amount to treat the subject. In one embodiment, the conditioned media fraction is derived from an isolated mesenchymal stem cell population having a defined antigen expression profile.

In still another aspect, the invention provides a method for preparing an MSC antigen presenting cell comprising contacting in vitro a naïve antigen presenting cell with a mesenchymal stem cell lysate or conditioned media, and allowing sufficient time for the naive antigen presenting cell to express on its surface an antigen or fragment thereof from the mesenchymal stem cell lysate or conditioned media, thereby generating an MSC antigen presenting cell. In a related method, the lysate or conditioned media may be fractionated and a resulting fraction may be contacted to the naïve antigen presenting cell. The fraction may be lipid-containing fraction, such as a vesicle containing fraction. The fraction may comprise lipids, organelles, polysaccharides, nucleic acids, or proteins, or a combination thereof such as a combination of lipids and nucleic acids. In one embodiment, the fraction comprises vesicles comprising RNA. Administration of the fraction rather than the entire lysate or conditioned media may reduce unnecessary contact with other components in the lysate or conditioned media.

In one embodiment, the mesenchymal stem cell lysate or conditioned media is generated from or using an isolated mesenchymal stem cell population. In one embodiment, the isolated mesenchymal stem cell population is an isolated mesenchymal stem cell population having a defined antigen expression profile. In one embodiment, the isolated mesenchymal stem cell population is prepared according to any of the foregoing methods. In one embodiment, the antigen presenting cell is a dendritic cell. In another embodiment, the antigen presenting cell is a B cell. The antigen presenting cell may be a macrophage or monocyte, an endothelial cell, or any other cell type having antigen presenting ability.

In a further aspect, the invention provides a method for modulating an immune response comprising administering to a subject in need thereof an MSC antigen presenting cell prepared according to any of the foregoing methods in an effective amount to modulate an immune response. In one embodiment, the immune response is an autoimmune response. In one embodiment, the immune response is down-regulated or redirected. The antigen presenting cell may be autologous to the subject in whom it is being administered, although it is not so limited (e.g., it may be sufficiently matched for transplant purposes). Additionally or alternatively, the mesenchymal stem cells (or conditioned media or lysates, in whole or fractionated) may be autologous to the antigen presenting cells and/or autologous to the subject, although other combinations are also contemplated by the invention.

In still another aspect, the invention provides a method for identifying a candidate mesenchymal stem cell comprising contacting a mesenchymal stem cell with an antigen-specific activated immune cell, and measuring antigen-specific activity of the antigen-specific activated immune cell prior to and after contact with the mesenchymal stem cell, wherein a reduction in antigen-specific activity as a result of contact with the mesenchymal stem cell identifies a candidate mesenchymal stem cell. In one embodiment, the mesenchymal stem cell is an isolated mesenchymal stem cell. In another embodiment, the mesenchymal stem cell is an isolated mesenchymal stem cell having a define antigen expression profile, which optionally may be prepared according to any of the foregoing methods.

These and other embodiments of the invention will be described in greater detail herein.

### BRIEF DESCRIPTION OF THE DRAWINGS

The Figures provided herein are not intended to be drawn to scale.

FIG. 1. Morphology and Immunophenotype of Infused Subpopulation of Marrow Stroma. Phase contrast images of adherent bone marrow cells (A) prior to immunodepletion. Cells were negatively immunodepleted against CD11b and CD45 using MACS. Fractions of CD11b+, CD45+ cells (B) and CD11b−, CD45− cells (C) are shown. (D) Immunocytochemistry of CD11b−, CD45− adherent bone marrow cell fractions showing positive reactivity to α-SMA. Flow cytometry analysis of adherent bone marrow cells after immunodepletion of CD11b+ and CD45+ cells. Histogram analysis of CD106 (E), CD90 (F), Flk-1 (G) and Sca-1 (H). Solid distributions represent cells stained with antibodies compared to unstained cells.

FIG. 2. Histological Changes in Ileal Tissue of Foxp3^{sf} Mice after MSC Transplant. Cross sections of distal ileum comparing wild-type (A) to Foxp3^{sf} mice treated with i.p. infusions of saline (B), T_{regs} (C), or MSCs (D). Inset images present higher magnification of boxed regions in original micrographs. Scale bars equal 250 μm (4× magnification). Data representative of 3 independent trials with a total of N=6 per group.

FIG. 3. MSC Treatment Reduces MLN Cellularity and Activated T Cell Number. Lymph nodes were harvested 7 days post-cell infusion of MSCs or Tregs and compared to untreated and wild-type nodes. (A) Gross histology of lymph nodes from mice. (B) After tissue harvesting, cellularity was determined using a Coulter Counter. (C) Lymph node cells were analyzed for CD4 and CD44 expression using flow cytometry. Data represent mean±s.e.m. of N=5 per group.

FIG. 4. Evaluation of eGFP+ MSC Engraftment in Foxp3^{sf} Mice. Tissues were harvested at 7 days post-treatment. Representative immunofluorescent images of the (A) distal ileum, (B) MLN, and (C) inguinal lymph node. eGFP is detected in red and DAPI is used as a nuclear counter-stain. (D) Semi-quantitative analysis of the number of eGFP+ cell clusters in MLNs and inguinal nodes.

FIG. 5. Alteration in Thymocyte Production and Serum Cytokine Levels after Cell Therapy. (A) Thymii from Foxp3^{sf} mice treated with vehicle or cells were analyzed for CD4 and CD8 expression using flow cytometry and compared to wild-type animals. Data representative of N=5 per group. Serum was analyzed for cytokines 1 week post-treatment by ELISA (B and C). Data represent mean±s.e.m. of 2 independent trials of a total of N=2 per group. *P<0.01.

FIG. 6. Cotransplantation of MSCs and T_{regs} Increases Splenic Engrafted T_{regs}. Splenocytes were harvested after 7 days of cell treatment and Foxp3+ cells were analyzed by flow cytometry. Wild-type C57Bl/6 mice have an endogenous T_{regs} compartment that is 5% of the spleen (A). Knockout mice have no Foxp3 expression, which is not altered by Foxp3− mMSCs (B). I.p. infusion of 3×10⁵ cells _{Tregs} resulted in 6% of splenocytes showing a positive reactivity for Foxp3. A 1:10 cell ratio of MSCs to T_{regs} led to an expansion of Foxp3+ splenocytes.

FIG. 7. Prevention of TNBS-Induced Colitis by MSC Transplantation. Kaplan-Meier analysis of cell-based transplantation strategies with intravenous delivery (A). Percentage of original body weight loss of mice over time (B). Semi-quantitative fecal occult blood testing of experimental groups (C). Kaplan-Meier analysis of MSC-CM prevention trial in colitis (D).

FIG. 8. Histopathological Analysis of Colitic Mice after MSC Infusion. Mesenteric lymph node (A) and transverse colon (B) of colitic mice 7 days post-cell therapy in the prevention trial. Lymph node cellularity and colon weight per length ratios are stated below the group names in units of [×106 cells/node] and [mg/cm], respectively. Representative microscopic specimens are shown for (C) saline, (D) fibroblast, and (E) MSC-treated colitic mice compared to (F) ethanol sham controls. Pathology scores are stated above the images. Histopathology analysis was performed on N=4 of each group.

FIG. 9. Increased Regulatory T Cell Number in Mesenteric Lymph Nodes after MSC Therapy. Flow cytometry of mesenteric lymph nodes for CD25 and Foxp3 expression. The graph represents one of two independent trials of N=4.

FIG. 10. Therapeutic Trial of MSC Transplantation in TNBS-Induced Colitis. (A and B) Survival analysis and (C) body weight loss in experimental mice after intravenous delivery of cells at two days disease onset.

FIG. 11. Mouse and Human MSCs Express Endogenous pTAs. Mouse stroma was isolated from wild-type mice, grown in culture for 7 days and separated by CD45 expression using MACS. (A) Endpoint RT-PCR analysis of pTA gene expression comparing whole bone marrow stroma, CD45+, and CD45− marrow stromal. Thymic tissue used as a positive control. Data are representative of 3 separate tests of mouse pTA expression using this pTA panel. (B) Immunofluorescence of AFP expression in CD45− cells. (C) Human pTA expression in MSCs after cryopreservation. (D) Quantitative RT-PCR analysis of AIRE and intestinal pTAs comparing UEA-1+ and UEA-1− thymic cells, CD45+ and CD45− fresh bone marrow cells, and long-term mouse MSCs after cryopreservation.

FIG. 12. UEA-1+ Bone Marrow Cells Express pTAs. (A) Bone marrow aspirates were analyzed by flow cytometry for CD45 expression and UEA-1 reactivity. (B) UEA-1 reactivity in cultured MSCs. (C) MACS-separated UEA-1+ bone marrow cells express pTAs.

FIG. 13. Immunohistochemistry of CD45-UEA-1 Localization in the Bone Marrow. Low and high power magnification of immunoperoxidase staining for UEA-1 (A,D), H&E stained (B,E), and CD45 (C,F) in wild-type mice. Two different fields of view are shown.

FIG. 14. Upregulation of Antigen Presentation Molecules after IFN-γ Stimulation. Purified MSCs were cultured in the presence of 20 ng/ml of IFN-γ for 24 hours and assessed for (A) PD-L1 and (B) MHC class II expression. Isotype control is a rat anti-IgG monoclonal antibody.

FIG. 15. Theory of Therapeutic Action Based on pTA Expression. The Figure shows a hypothesis of AIRE-dependent translation and presentation of pTAs. This mechanism is hypothesized to lead to immunosuppression by either: (a) anergizing self-reactive T cells, (b) transfer of antigens to DCs to anergize T cells, or (c) indirect generation of tolerizing DCs and the generation of regulatory T cells in situ. pTA, peripheral tissue antigen; mTEC, medullary thymic epithelial cell; DC, dendritic cell; LNSC, lymph node stromal cell.

FIG. 16 shows relative expression of various pTAs in five independent murine BMSC colonies.

FIG. 17 shows a graph indicating relative viability of wild-type AIRE BMSCs versus AIRE-deficient BMSCs.

FIG. 18 shows a graph indicating cell proliferation data measured for wild-type and AIRE-deficient murine CD45− BMSCs that were co-cultured with splenocytes in the presence of anti-CD3e in a first experiment.

FIG. 19 shows a graph indicating cell proliferation data measured for wild-type and AIRE-deficient murine CD45− BMSCs that were co-cultured with splenocytes in the presence of anti-CD3e in a second experiment.

FIG. 20 shows a graph indicating the relative levels of various markers in wild-type BMSCs as compared to the levels present in AIRE-deficient BMSCs.

FIG. 21 shows dotplot data indicating that both PDGF-β and gp38 are expressed in CD45− murine BMSCs.

FIG. 22 shows histograms of CFSE dilution as a measure of proliferation of OVA-specific T cells (OT-I cells) that have been cultured in different conditions. OT-I cells cultured alone or with wild-type antigen presenting cells (dendritic cells, CD45+ bone marrow cells, or CD45− bone marrow stromal cells) for 60 hours with or without prior OVA pre-incubation for 24 hours to the antigen presenting cells.

FIG. 23 OT-I cells cocultured with CD45+ or CD45− bone marrow cells that were derived from iFABP-tOVA mice. The left histograms show CFSE dilution in OT-I cells after coculture for the transgenic marrow cells. The right shows a bar graph quantifying the results of FIGS. 22 and 23 with a table below stating the p-statistic of the experimental groups tested. Arrows highlight comparisons of statistical significance.

FIG. 24. Histograms of OT-I proliferation after coculture with dendritic cells that were pre-incubated with no antigen (top row), purified OVA peptide (middle row), or with various concentrations of lysates from CD45− marrow stromal cells isolated from iFABP-tOVA mice (bottom row). The ratio of lysate from an equivalent stromal cell number to the number of dendritic cells is shown above.

FIG. 25. Generation of Foxp3+ Splenocytes after MSC Coculture is Dependent on CD11b+ Cells. Splenocytes were cultured for 5 days with or without mesenchymal cells at to a 1:10 ratio (mesenchymal cell:splenocyte) and analyzed for Foxp3 expression using flow cytometry. Dotplots gated on CD4 expression of unfractionated splenocytes cultured with (A) IL-2 alone, (B) fibroblasts and IL-2, or (C) MSCs and IL-2. The graph represents one of five independent trials. (D) Dotplot of CD11b+ depleted splenocytes cocultured with MSCs and IL-2. (E) Results of five independent trials comparing percentage of CD25+ Foxp3+ cells to coculture conditions.

FIG. 26. Generation of Foxp3+ Splenocytes after MSC Coculture is Independent of Enhanced Proliferation of CD4+ CD25+ T Cells or Conversion of Naïve Cells to a Suppressor Phenotype. Dotplots gated on CD4 expression of CD4+ CD25− splenocytes were cultured for 5 days (A) with or (B) without MSCs and analyzed for Foxp3 expression using flow cytometry. The graph represents one of three independent trials of N=4. (C) Total Foxp3+ cell number after coculture of MACS separated CD25+ cells with no cell, MSCs, or in the presence of rhIL-2 for 5 days. After culture, cells were counted and analyzed for CD25+ Foxp3+ cells. The product of cell number and percentage of CD25+ Foxp3+ cell number is plotted as the absolute cell number for each condition.

FIG. 27. Adoptive Transfer of CD11b+ cells after MSC coculture confers increase in regulatory T cell number. Left shows the schematic of the coculture regimen. CD11b+ splenocytes were cocultured at a 1:1 ratio with a mesenchymal cell in IL-2 supplemented medium and were isolated after coculture using magnetic bead separation. These CD11b+ cells were transferred into wells with whole splenocytes at different ratios (ratio of whole splenocytes to CD11b+ cells) in IL-2 medium and after 5 days of coculture, regulatory T cell frequency was assessed. Shown on the right are dotplots of CD25+ Foxp3+ cells that are increased in a dose dependent manner as a function of the number of transferred CD11b+ cells.

FIG. 28. The creation of a new tolerogenic cell type: adoptive Transfer of CD11b+ cells after MSC coculture leads to survival benefit in colitic mice. CD11b+ splenocytes were cocultured at a 1:1 ratio with a mesenchymal cell in IL-2 supplemented medium and were isolated after coculture using magnetic bead separation. These CD11b+ cells were transferred i.v. into mice directly before they had been administered TNBS to induce colitis. Different numbers of CD11b+ cells were transferred to determine if there is a dose-dependent response. Shows is a bar graph of the one week survival of colitic mice.

FIG. 29. IFN-γ downregulates AIRE and pTA expression. Purified MSCs were cultured in the presence of 20 ng/ml of IFN-γ for 24 hours and assessed for AIRE and other pTAs by quantitative RT-PCR.

### DETAILED DESCRIPTION OF THE INVENTION

The invention is based in part on the finding that mesenchymal stem cells are able to induce tolerance to self antigens. This finding is based in part on the additional finding that mesenchymal stem cells express a variety of antigens, including as discussed herein peripheral tissue antigens. The ability to express peripheral tissue antigens allows mesenchymal stem cells to induce tolerance in immune cells, including T cells, and thereby modulate immune responses such as aberrant immune responses.

It has been further discovered according to the invention that mesenchymal stem cells may be differentiated based on their antigen expression profiles. That is, mesenchymal stem cells can be physically fractionated and thus isolated according to their antigen expression profiles in order to enrich for cells that express more or less of one or more particular antigens, or for cells that express a particular antigen repertoire. The antigen repertoires may be specific for a particular tissue and thus such cells may be suitable for inducing tolerance to self antigens in such tissues. Although not intending to be bound by any particular theory, it is contemplated that mesenchymal stem cells induce tolerance by inhibiting and/or deleting immune cells reactive to self antigens, akin to tolerance mechanisms that occur in the thymus.

The invention therefore exploits these novel and unexpected findings relating to antigen expression by mesenchymal stem cells. In this way, the invention contemplates and provides methods for preparing isolated mesenchymal stem cells (or populations) having defined antigen expression profiles, and using such populations both in vivo and in vitro. The invention further contemplates use of mesenchymal stem cell products such as but not limited to conditioned media from a mesenchymal stem cell culture, lysates of mesenchymal stem cells, as well as fractions thereof. Such fractions may be generated by physical, chemical, enzymatic, or other parameter that separates components in the starting population. As an example, the fractionation may generate a lipid-containing fraction and a substantially lipid-free fraction.

Various aspects and embodiments of the invention relate to the analysis, manipulation, prophylactic or therapeutic use, and/or screening of mesenchymal stem cells. A mesenchymal stem cell is a progenitor cell having the capacity to differentiate into neuronal cells, adipocytes, chondrocytes, osteoblasts, myocytes, cardiac tissue, and other endothelial and epithelial cells. (See Wang, Stem Cells 2004; 22(7);1330-7; McElreavey; 1991 Biochem Soc Trans (1); 29s; Takechi, Placenta 1993 March/April; 14 (2); 235-45; Takechi, 1993; Kobayashi; Early Human Development; 1998; Jul. 10; 51 (3); 223-33; Yen; Stem Cells; 2005; 23 (1) 3-9.) These cells may be defined phenotypically by gene or protein expression. These cells have been characterized to express (and thus be positive for) one or more of CD13, CD29, CD44, CD49a, b, c, e, f, CD51, CD54, CD58, CD71, CD73, CD90, CD102, CD105, CD106, CDw119, CD120a, CD120b, CD123, CD124, CD126, CD127, CD140a, CD166, P75, TGF-bIR, TGF-bIIR, HLA-A, B, C, SSEA-3, SSEA-4, D7 and PD-L1. These cells have also been characterized as not expressing (and thus being negative for) CD3, CD5, CD6, CD9, CD10, CD11a, CD14, CD15, CD18, CD21, CD25, CD31, CD34, CD36, CD38, CD45, CD49d, CD50, CD62E, L, S, CD80, CD86, CD95, CD117, CD133, SSEA-1, and ABO.

Thus, mesenchymal stem cells can be characterized phenotypically and/or functionally according to their differentiative potential.

In a preferred embodiment, the mesenchymal stem cells are derived from bone marrow and are adherent and are negative for both cell surface expression of CD11b and CD45. These cells may be additionally characterized in some embodiments as CD105+ (SH-2+), CD73+ (SH-3+ and SH-4+), CD34−, and CD14−.

Mesenchymal stem cells may also be harvested and isolated from other cells of the bone marrow, for instance, using osmotic methods since it has been found according to the invention that mesenchymal stem cells are more resilient to osmotic shock than are other cells of the bone marrow. Therefore, one method for isolating mesenchymal stem cells from bone marrow is to expose a bone marrow cell population to water or a low ionic strength aqueous solution for brief periods of time, followed by harvest of the stem cells.

Mesenchymal stem cells from umbilical cord matrix is defined as an adherent cell population having a fibroblastoid phenotype and the expression of mesenchymal markers CD105^{+(SH-2)}, CD73^{+(SH3)} and CD34⁻, CD45⁻. These cells also express Oct-4 and

Mesenchymal stem cells may be harvested from a number of sources including but not limited to bone marrow, blood, periosteum, dermis, umbilical cord blood and/or matrix, and placenta. Methods for harvest of mesenchymal stem cells from the bone marrow are described in greater detail in the Examples. Reference can also be made to U.S. Pat. No. 5,486,359 for other harvest methods that can be used in the present invention.

As used herein, it is to be understood that aspects and embodiments of the invention relate to cells as well as cell populations, unless otherwise indicated. Thus, where a cell is recited, it is to be understood that a cell population is also contemplated unless otherwise indicated.

As used herein, an isolated mesenchymal stem cell is a mesenchymal stem cell that has been physically separated from its natural environment, including physical separation from one or more components of its natural environment. Thus, an isolated cell or cell population embraces a cell or a cell population that has been manipulated in vitro or ex vivo. As an example, isolated mesenchymal stem cells may be mesenchymal stem cells that have been physically separated from at least 50%, preferably at least 60%, more preferably at least 70%, and even more preferably a least 80% of the cells in the tissue from which the mesenchymal stem cells are harvested. In some instances, the isolated mesenchymal stem cells are present in a population that is at least 40%, at least 50%, at least 60%, at least 70%, at least 80%, at least 85%, at least 90%, at least 95%, at least 96%, at least 97%, at least 98%, at least 99%, or 100% mesenchymal stem cells as phenotypically and/or functionally defined herein. Preferably the ratio of mesenchymal stem cells to other cells is increased in the isolated preparation as compared to the starting population of cells.

Mesenchymal stem cells can be isolated using methods known in the art, e.g., from bone marrow mononuclear cells, umbilical cord blood, adipose tissue, placental tissue, based on their adherence to tissue culture plastic. For example, mesenchymal stem cells can be isolated from commercially available bone marrow aspirates. Enrichment of mesenchymal stem cells within a population of cells can be achieved using methods known in the art including but not limited to FACS.

Commercially available media may be used for the growth, culture and maintenance of mesenchymal stem cells. Such media include but are not limited to Dulbecco's modified Eagle's medium (DMEM). Components in such media that are useful for the growth, culture and maintenance of mesenchymal stem cells include but are not limited to amino acids, vitamins, a carbon source (natural and non-natural), salts, sugars, plant derived hydrolysates, sodium pyruvate, surfactants, ammonia, lipids, hormones or growth factors, buffers, non-natural amino acids, sugar precursors, indicators, nucleosides and/or nucleotides, butyrate or organics, DMSO, animal derived products, gene inducers, non-natural sugars, regulators of intracellular pH, betaine or osmoprotectant, trace elements, minerals, non-natural vitamins. Additional components that can be used to supplement a commercially available tissue culture medium include, for example, animal serum (e.g., fetal bovine serum (FBS), fetal calf serum (FCS), horse serum (HS)), antibiotics (e.g., including but not limited to, penicillin, streptomycin, neomycin sulfate, amphotericin B, blasticidin, chloramphenicol, amoxicillin, bacitracin, bleomycin, cephalosporin, chlortetracycline, zeocin, and puromycin), and glutamine (e.g., L-glutamine).

Mesenchymal stem cell survival and growth also depends on the maintenance of an appropriate aerobic environment, pH, and temperature.

As one example, mesenchymal stem cells may be prepared as follows. Bone marrow cells can be cultured using Dulbecco's modified Eagle's medium supplemented with 10% fetal calf serum, 100 U/ml penicillin, 100 μg/ml streptomycin, 0.1 mM non-essential amino acids and 1 ng/ml of basic fibroblast growth factor (Life Technologies, Rockville, Md.). After 4 days of culture, non-adherent cells can be removed by washing with PBS. Monolayers of adherent cells are then cultured with medium changes 2-3 times per week. Cells can be passaged using 0.25% trypsin/0.1% EDTA and subcultured at a density of 5×10₃ cells/cm². Mesenchymal stem cells can be maintained using methods known in the art (see, e.g., Pittenger et al., Science, 284:143-147 (1999)).

Additionally, mesenchymal stem cells of the invention may be cryopreserved for any length of time. It has been discovered according to the invention that mesenchymal stem cells demonstrate a robust antigen expression capability allowing them to maintain a continuous and consistent antigen expression profile throughout culture and various passages and also throughout freezing and thawing processes regardless of the length of culture and/or storage.

The invention therefore provides compositions comprising the isolated mesenchymal stem cells having defined antigen expression profiles of the invention. Such compositions may or may not be cryopreserved. Such compositions may be pharmaceutically acceptable such that they are suitable for administration to a subject for diagnostic, prophylactic or therapeutic purpose.

The invention further contemplates the preparation, analysis, and use of isolated mesenchymal stem cells having a defined antigen expression profile. As used herein, an isolated mesenchymal stem cell having a defined antigen expression profile is an isolated mesenchymal stem cell that expresses and/or fails to express one or more antigens, and optionally may express one or more antigens at a particular level. These cells may be naturally occurring and physically separated from other cells based on their antigen expression profile. This can be accomplished using fluorescence activated cell sorting (FACS) where cells are physically separated from each other based on expression (or lack thereof) of one or more antigens. This can also be accomplished through the use of panning which involves contacting cells to a solid support (usually a petri dish) having an antigen-specific antibody attached thereto and allowing cells that express the particular antigen to bind to the solid support. Cells that express the antigen and therefore bind to the plate may be subsequently removed from the plate and harvested. In this way, cells may be separated into those that express and those that do not express the antigen. In a similar manner, cells may be labeled with antigen-specific antibody and then subsequently contacted with magnetic beads conjugated to secondary isotype specific antibodies. In this way, cells that express the particular antigen are bound to the magnetic beads and then separated from the starting cell population through magnetic separation. Isolation of cells having a defined antigen expression profile can also be accomplished by targeting and killing cells that express one or more antigens by labeling such cells with antibodies and incubating them with complement in order to lyse antibody bound cells.

Alternatively, isolated mesenchymal stem cells having a defined antigen expression profile may be mesenchymal stem cells that are genetically engineered to express one or more antigens, optionally at particular levels. Methods for genetically engineering cells to express one or more nucleic acids and thus antigens are known in the art and discussed in greater detail herein.

An antigen expression profile therefore is a characterization of a cell or cell population based on the type of antigens expressed (and alternatively, not expressed) by the cell and optionally the level of expression of such antigens (e.g., compared to the expression level of housekeeping or other constitutively expressed genes). The antigen expression profile may be a single antigen expression profile (i.e., characterization of a cell based on whether it expresses a single antigen and optionally the level of expression of that single antigen), or it may be an antigen expression profile based on a plurality of antigens.

Antigen expression as referred to herein typically refers to cell surface expression of antigens. However, in the case of mesenchymal stem cells gene expression levels (i.e., mRNA expression) may be used as a surrogate marker or indicator for cell surface expression of antigens.

Various aspects and embodiments of the invention comprise determining the level of expression or inducing the expression of one or more peripheral tissue antigens in a cell or cell population. These antigens are also known in the art as peripheral tissue regulated antigens. These antigens are typically self antigens. These antigens are expressed in one or more peripheral tissues and are also ectopically expressed by medullary epithelial cells of the thymus either continuously or sporadically. This latter expression pattern and profile serves to induce tolerance of immune cells to self peripheral tissues. One category of such antigens are those that are transcriptionally upregulated by the Aire gene product. Examples include IL-9, ccl17, ccl22, cxcl9, tap1, ctsL, H2-Mα, mecl 1, ccl19, gilt, cxcl10, erp57, H2-Mβ2, H2-Oa, bip, li, ctsS, bax, H20Mβ1, H2-Oβ, IL-12a, IL-4, ccl25, CTLA-4, abhydrolase domain containing 6, activity regulated cytoskeletal-associated protein, aldose reductase, alkB, alkylation repair homolog 5 (E. coli), α-1 microglobulin/bilunin precursor, amelogenin, argininosuccinate lyase, ATP-binding cassette, Burkitt lymphoma receptor 1, sub-family C (CFTR), casein alpha, chemokine (C-X-C motif) receptor 7, cystic fibrosis transmembrane conductance regulator homolog, cryptdin related sequence 2, cytochrome P450 1a2, deltex 4 homolog (Drosophila), desmoglein 1a (Dsg1a, involved in pemphigus foliaceus), EGF-like-domain, multiple 6, FAD-dependent oxidoreductase domain containing 2, fatty acid binding protein, gamma-casein precursor, GH regulated TBC protein 1, glucosaminyl (N-acetyl) transferase 1, core 2, glucose dependent insulinotropic polypeptide, glutamate receptor, ionotropic, NMDA2C (epsilon 3), Grin2C, Gulo, hemoglobin y (beta like embryonic chain), 3-hydroxyanthranilate 3,4-dioxygenase, insulin-like growth factor II, inter-alpha inhibitor H3 chain, intestinal trefoil factor, lactotransferrin, ladinin 1 (Lad1, involved in linear IgA dermatosis), lung-inducible neuralized-related C3HC4 RING domain protein, major urinary protein 1, major urinary protein 3, major urinary protein 4, mast cell protease 2, mesoderm development candidate 2, mucin 6, necdin-like 2, neurotoxin homologue, neutrophilic granule, neutrophil cytosolic factor 4, NMDA receptor 2C (involved in systemic lupus erythematosis), NUAK family, SNF1-like kinase, 2, oxytocin, phosphofructokinase, liver, B-type, preproinsulin II, preproneuropeptide y, proline rich 14, prostaglandin D, Purkinje cell protein 4, retinoic acid receptor responder (tazarotene induced) 1, RIKEN cDNA 9930023K05 gene, Ras-related associated with diabetes, Rrad, ryanodine receptor 3, S100 calcium binding protein A9, salivary protein 1, salivary protein 2, serine protease (BSSP), spermine binding protein, TMEM9 domain family, member B, transmembrane 7 superfamily member 4, and ubiquitin conjugated enzyme E2N. Reference can also be made to Anderson et al. Science 298:1395 (2002) and Gardner et al. Science 321(5890):843-847 (2008) (Table SI) for additional information relating to and examples of peripheral tissue antigens. The teachings in these references relating to examples of pTAs are incorporated by reference herein.

Antigens useful in the invention may be nucleic acids and proteins, and the like. Any pTA may be transferred from for example a mesenchymal stem cell (or lysate or conditioned media) to an antigen presenting cell such as a dendritic cell as a protein, a protein fragment, or a nucleic acid that encodes the protein. The nucleic acid may be a DNA or an RNA.

In still other aspects and embodiments of the invention methods are contemplated whereby transfer of mesenchymal stem cell constituents occurs between mesenchymal stem cells and antigen presenting cells. The mesenchymal cell may be used to transfer proteins or fragments thereof (as discussed above), nucleic acids or fragments thereof (as discussed above), lipids including vesicles or microvesicles that themselves are complexed to cellular components, organelles or fragments thereof, carbohydrates, polysaccharides, and the like. In some instances, the transfer occurs by way of a complex formed between lipids and other cellular components such as nucleic acids (e.g., mRNA or siRNA). Such complexes may take the form of a vesicle that surrounds a cellular component, but they may also be complexes in which the cellular component is still exposed to the environment, wholly or partially. Thus, in some instances, the invention contemplates fractionation of lysates, conditioned media and the like in order to isolate one or more of these components for presentation to antigen presenting cells. It is also contemplated that these components once isolated may be complexed with exogenous lipids or other carriers in order to facilitate uptake by antigen presenting cells.

The invention provides a cell bank that comprises at least one and preferably more stored samples of isolated mesenchymal stem cells. Preferably the antigen expression profile for such cells is known and thus such cells have defined antigen expression profiles. Even more preferably, the antigen expression profile is a peripheral tissue antigen expression profile. These cells may be generated by any of the methods of the invention relating thereto. The bank may comprise only one aliquot of any given mesenchymal stem cell population or it may contain two or more aliquots of the same cell population.

The invention further contemplates a bank that comprises one or more samples comprising cell lysates of isolated mesenchymal stem cells, whether or not such cells have been phenotypically characterized (and thus have a known defined antigen expression profile). The bank may comprise samples of cells as well as samples of lysates derived from the same cells. In preferred embodiments, the lysates are generated from mesenchymal stem cells that have been phenotypically characterized (and thus have a known defined antigen expression profile). The sample may be a lysate fraction, such as but not limited to a lipid containing fraction of the lysate.

The invention further contemplates a bank that comprises one or more samples of mesenchymal stem cell conditioned media, as described herein. The bank may further comprise samples of cells as well as samples of mesenchymal stem cell conditioned media, and may further comprise samples of lysates derived from the same cells. The sample may be a conditioned media fraction, such as but not limited to a lipid containing fraction of the conditioned media.

In one embodiment, the bank may further comprise a database such as an electronic database (e.g., a computer database) for storing information relating to the stored samples. The database may comprise an information record for each sample and this informational record may minimally contain a field that lists the antigen expression profile of the sample. The information record may further comprise information about the protocol (including reagents) used to generate the sample. The information record may also identify the source of the sample including tissue and subject. The database may comprise one or more fields and/or one or more subfields.

Mesenchymal stem cell lysates may be prepared by any lysis method known in the art provided that the resulting lysate is not toxic to cells. These methods include chemical and/or mechanical methods such as osmotic shock, ultrasound, and shearing of cells. The method may alternatively be an enzymatic method using for example an enzyme that is physically separable from the resulting lysate. The lysate may be concentrated, filtered, or manipulated in other ways that do not impact its antigen content. In some important embodiments, the lysate is fractionated according to lipid content, and accordingly a lipid containing fraction is generated. As used herein, a lipid containing fraction is a fraction that contains a greater proportion (e.g., w/w or w/v) of lipid constituents than does the starting population from which it derived. Thus, the fraction need not contain all the lipid present in the starting population although it should be enriched in such lipid constituents.

The invention further contemplates exploiting the antigen repertoire of mesenchymal stem cells in order to render other (naïve) antigen presenting cells capable of inducing tolerance also. This aspect of the invention contemplates lysing mesenchymal stem cells, extracting and/or harvesting the resulting lysate, and exposing antigen presenting cells to such lysate (or a fraction thereof) for an appropriate period of time to allow the antigen presenting cell to uptake antigens within the lysate. Thereafter or simultaneously, the antigen presenting cells are allowed to process such antigens and express such antigens and/or fragments thereof on their surface. The resulting antigen presenting cell is referred to herein as an MSC antigen presenting cell (or MSC APC), as it is an APC that expressed antigens derived from an MSC.

The naïve antigen presenting cells may be dendritic cells, B cells, macrophages, monocytes, endothelial cells, or any other antigen presenting cell, and may be harvested from any appropriate tissue.

The invention further contemplates use of such MSC APC in the same in vitro and in vivo methods contemplated for the isolated mesenchymal stem cells of the invention. For example, the MSC APC may be administered to a subject having or at risk of developing an aberrant immune response such as an autoimmune response or a graft-versus-host immune response, in order to induce tolerance to self antigen in any self-reactive immune cells including self-reactive T cells. In these and other aspects and embodiments of the invention, it should be understood that the invention contemplates modulating immune responses in order to balance such responses and reduce deleterious side effects. IN these and other methods, the active agents including but not limited to the MSC APC may be administered locally or systemically.

The MSC APC may be cryopreserved and/or stored in a cell bank as described herein, as may be the MSC lysate (or a fraction thereof) used to generate the cells.

The invention contemplates genetically engineering mesenchymal stem cells to express one or more antigens, preferably peripheral tissue antigens. This can be accomplished using methods known in the art. Expression vectors to be introduced into mesenchymal stem cells will generally include the pertinent sequence, i.e., nucleotide sequences that encode the peripheral tissue antigen, and transcriptional and translational control sequences such as promoters, enhancers, poly A sequences, termination sequences and the like. In some instances, two or more coding sequences (i.e., two or more sequences each coding for a peripheral tissue antigen) may be included in the vector, preferably with an IRES or functionally equivalent sequence located therebetween. The cells being so transduced or transfected may not naturally express one or more of the antigens encoded by the expression vectors or may not express them at suitable levels.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined to each other when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA) encoding an antigen of interest. That heterologous DNA (RNA) is placed under operable control of transcriptional elements to permit the expression of the heterologous DNA in the host cell.

Preferred systems for mRNA expression in mammalian cells are those such as pRc/CMV and pcDNA3.1 (available from Invitrogen, Carlsbad, Calif.) that contain a selectable marker such as a gene that confers G418 resistance (which facilitates the selection of stably transfected cell lines) and the human cytomegalovirus (CMV) enhancer-promoter sequences. Additionally, suitable for expression in primate or canine cell lines is the pCEP4 vector (Invitrogen), which contains an Epstein Barr virus (EBV) origin of replication, facilitating the maintenance of plasmid as a multicopy extrachromosomal element. Another expression vector is the pEF-BOS plasmid containing the promoter of polypeptide Elongation Factor 1α, which stimulates efficiently transcription in vitro. The plasmid is described by Mishizuma and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for E1 and E3 proteins (J. Clin. Invest. 90:626-630, 1992). The use of the adenovirus as an Adeno.P1A recombinant is disclosed by Warnier et al., in intradermal injection in mice for immunization against P1A (Int. J. Cancer, 67:303-310, 1996). Recombinant vectors including viruses selected from the group consisting of adenoviruses, adeno-associated viruses, poxviruses including vaccinia viruses and attenuated poxviruses such as ALVAC, NYVAC, Semliki Forest virus, Venezuelan equine encephalitis virus, retroviruses, Sindbis virus, Ty virus-like particle, other alphaviruses, VSV, plasmids (e.g., “naked” DNA), bacteria (e.g., the bacterium Bacille Calmette Guerin, attenuated Salmonella), and the like can be used in such delivery, for example, for use as a vaccine.

An MSC-CM composition can be prepared by culturing a mesenchymal stem cell population for a period of time and then harvesting the culture media apart from the cells. The population may one that has been passaged or one that has just been isolated and cultured. Preferably, it has been passaged and more preferably it is between passage 4-7. The mesenchymal stem cells may be cultured at a density of about 1×10⁵ to 1×10⁷ cells, e.g., about 1×10⁵ to 1×10⁶ cells, 1×10⁶ to 1×10⁷ cells, 1×10⁶ to 9×10⁶ cells, 1×10⁶ to 8×10⁶ cells, 1×10⁶ to 7×10⁶ cells, 1×10⁶ to 6×10⁶ cells, 1×10⁶ to 5×10⁶ cells, 1×10⁶ to 4×10⁶ cells, 1×10⁶ to 3×10⁶ cells, and 1×10⁶ to 2×10⁶ cells.

In some embodiments, an MSC-CM is prepared as follows: (1) wash 70-80% confluent mesenchymal stem cells thoroughly with phosphate buffered saline (PBS); (2) Culture mesenchymal stem cells for about 12, 24, 36, or 48 hours, e.g., 24 hours in an appropriate volume of serum free culture medium containing DMEM, or an equivalent thereof, supplemented with 0.05% bovine serum albumin (BSA) in a suitable vessel, e.g., a T175 cm² flask, with each vessel/flask at 80% confluency, equivalent to about 5×10³ 15 cells/cm²; and (3) Collect MSC culture media from (2).

The collected MSC-CM can be concentrated, e.g., using methods known in the art, for example, ultrafiltration units with a 3 kD cutoff (AMICON Ultra-PL 3, Millipore, Bedford, Mass., USA). For example, the MSC-CM can be concentrated at least 2-fold to 10-fold, 10-fold to 20-fold, 20-fold to 30-fold, 30-fold to 49-fold, and above. As one example, an MSC-CM is concentrated 25-fold. In some embodiments, the MSC-CM comprises culture medium containing DMEM supplemented with 0.05% bovine serum albumin (BSA). In some embodiments, the MSC-CM composition does not contain any animal serum or other animal products. In some embodiments, the MSC-CM composition comprises PBS. Alternatively, the MSC-CM is provided in lyophilized form. In some embodiments, an MSC-CM can be fractionated by size or by charge. It is to be understood that the conditioned media may be processed and/or manipulated in any number of ways prior to and/or after ultracentrifugation, as the case may be.

In some embodiments, for example, an MSC-CM can be fractionated into heparin sulfate binding and non heparin binding fractions. For example, in heparin sulfate fractionation experiments, a concentrated MSC-CM can be passed over a heparin column, or other columns e.g., an ion-exchange, size, reverse-phase or other chromatographic separation methods per vendor's instructions. Flow-through and eluted fractions can then be collected separately. The eluted fractions (i.e., the heparin-binding fraction) can then be collected and optionally concentrated, as described above. In some embodiments, an MSC-CM composition is at least 50%, 60%, 70%, 80%, 90%, and 100% free of non-heparin binding material.

The invention further contemplates the use of the isolated mesenchymal stem cells, the MSC APC, the MSC-CM conditioned media, and/or the MSC lysates, alone or in any combination for the prevention or treatment of aberrant immune responses and/or conditions resulting therefrom. Subjects to whom these cellular and/or acellular compositions may be administered include those at risk of developing aberrant immune responses (and the conditions resulting therefrom) based on for example a genetic predisposition, or subjects presently having such immune responses.

As used herein, an aberrant immune response is one that is upregulated compared to immune responses in a normal subject population. In some important embodiments, the immune response is directed to a self antigen (i.e., an antigen that is encoded in the genome of the subject being treated). The normal subject population is one that does not possess such anti-self immune reactivity, except as may occur for example in cancer immunosurveillance.

Thus, the methods provided herein aim to reduce, diminish, control or completely eliminate such aberrant immune responses. Subjects in need of such immunomodulation include those having or those at risk of developing autoimmune diseases, those having or at risk of graft-versus-host disease, and the like.

The invention contemplates that the cells and/or lysates to be administered to a particular subject are selected based on the antigen expression profile thereof. For example, the invention contemplates administering to a subject having colitis an isolated mesenchymal stem cell population having a defined antigen expression profile that comprises one or more gut antigens. In this way, the invention provides a personalized and customized treatment for a subject based on the disease and antigens triggering the disease.

Preventing a disease means reducing the likelihood that the disease manifests itself and/or delaying the onset of the disease. Treating a disease means reducing or eliminating the symptoms of the disease.

One aspect of the invention relates to the treatment of autoimmune diseases. Examples of autoimmune diseases include but are not limited to multiple sclerosis, inflammatory bowel disease including Crohn's Disease and ulcerative colitis, rheumatoid arthritis, psoriasis, type I diabetes, uveitis, Celiac disease, pernicious anemia, Srojen's syndrome, Hashimoto's thyroiditis, Graves' disease, systemic lupus erythamatosis, acute disseminated encephalomyelitis, Addison's disease, Ankylosing spondylitis, Antiphospholipid antibody syndrome, Guillain-Barre syndrome, idiopathic thrombocytopenic purpura, Goodpasture's syndrome, Myasthenia gravis, Pemphigus, giant cell arteritis, aplastic anemia, autoimmune hepatitis, Kawaski's disease, mixed connective tissue disease, Ord' throiditis, polyarthritis, primary biliary sclerosis, Reiter's syndrome, Takaysu's arteritis, vitiligo, warm autoimmune hemolytic anemia, Wegener's granulomatosis, Chagas' disease, chronic obstructive pulmonary disease, and sarcoidosis.

In important embodiments, the autoimmune disease is an inflammatory bowel disease including but not limited to colitis and Crohn's disease.

A subject at risk of developing an autoimmune disease includes one who is genetically predisposed to the disease. Such a subject may have one or more family members that are afflicted with the disease.

When administered, the compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents.

The preparations of the invention are administered in effective amounts. An effective amount is that amount of a pharmaceutical preparation that alone, or together with further doses, stimulates the desired response. The absolute amount will depend upon a variety of factors, including the material selected for administration, whether the administration is in single or multiple doses, and individual patient parameters including age, physical condition, size, weight, and the stage of the disease. These factors are well known to those of ordinary skill in the art and can be addressed with no more than routine experimentation.

The compositions may be administered systemically (e.g., through intravenous injection) and/or locally.

The invention further contemplates screening assays employing mesenchymal stem cells. Such assays are directed at identifying mesenchymal stem cell populations that are able to induce tolerance of specific immune cells (i.e., immune cells from particular autoimmune diseases, or from a subject having graft versus host disease, and the like). In this way, mesenchymal stem cell populations can be generated that are characterized functionally rather than phenotypically as described herein. The invention contemplates that some mesenchymal stem cells will be better able to induce tolerance to particular antigens based on their antigen expression profiles.

One example of a screening assay involves contacting a mesenchymal stem cell with an antigen-specific activated immune cell and measuring antigen-specific activity of the antigen-specific activated immune cell prior to and after contact with the mesenchymal stem cell. A reduction in antigen-specific activity as a result of contact with the mesenchymal stem cell identifies the mesenchymal stem cell as a potential candidate for clinical use. The immune cell may be a T cell. The mesenchymal stem cell may be an isolated mesenchymal stem cell. The mesenchymal stem cell may be an isolated mesenchymal stem cell having a define antigen expression profile.

Assays for measuring the antigen-specific activity may include the ability of the cell to lyse a target cell that expresses the antigen, optionally measured by the release of cellular contents into the supernatant including a radioactive or fluorescent marker.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### EXAMPLES

### Example 1

Materials and Methods

Mice. C57Bl/6 mice between 4 to 6 weeks of age were purchased from Charles River Laboratory. Foxp3^{sf} mice were purchased from Jackson Laboratory. Animals were maintained in a light-controlled room (12-h light-dark cycle) at an ambient temperature of 25° C. with chow diet and water ad libitum. The animals were cared for in accordance with the guidelines set forth by the Committee on Laboratory Resources, National Institutes of Health. All experimental procedures performed were approved by Subcommittee on Research Animal Care and Laboratory Animal Resources of Massachusetts General Hospital. Foxp3^{sf} mice were housed and used in a pathogen-free facility at Shriners Hospitals for Children in accordance with all applicable guidelines.

Antibody and Reagents. The following antibodies used for flow cytometry were purchased from Pharmingen: CD4-APC, CD44-FITC, CD25-PE, CD8-FITC, CD106-FITC, Flk-1-PE, CD90-FITC, and Sca-1-FITC. Biotinylated antibodies to CD4 and CD25 were purchased from eBiosciences. Streptavidin microbeads, CD45 and CD11b microbeads along with magnetic columns were purchased from Milenyi Biotec. For immunocytochemistry, anti-mouse α-SMA was purchased from Santa Cruz Biotechnology. MSC expansion medium consisted of alpha-MEM without deoxyribonucleosides and ribonucleosides (Gibco), 10% lot selected FBS (Atlanta Biologicals), 100 U/ml penicillin (Sigma), and 100 μg/ml streptomycin (Sigma).

Isolation and Culture of Bone Marrow-Derived MSCs. Bone marrow was harvested from wild-type mice after euthanization. Tibias and femurs were dissected and the marrow space was flushed with MSC expansion medium using a 23 gauge needle. Bone marrow plugs were collected on ice, dissociated by repeated passage through an 18 gauge needle and passed through a 70 μm filter to remove bony spicules and debris. Approximately 50×10⁶ bone marrow cells were plated on a 100 mm² tissue culture dish and cultured for 3 days to allow for differential adhesion of stromal cells. Non-adherent cells were aspirated on day 3 and the adherent population was cultured in MSC expansion medium for a subsequent 4-10 days to achieve the maximal number of colony forming unit-fibroblast prior to initial passage. Cells were passaged using 0.1% trypsin/0.1% EDTA, and subcultured at a density of 5×10³ cells/cm². Prior to transplantation, stromal cells were then depleted of CD11b and CD45 cells using magnetic activated cell sorting (MACS) per vendor's instructions. Enhanced green fluorescent protein (eGFP)-MSCs were kindly donated by the Center for Gene Therapy at Tulane University and grown in MSC expansion medium. All cultures were used between passages 2-5.

Isolation and Analysis of Cells from Lymph Nodes and Thymii. Lymphoid organs were dissected from experimental mice and dissociated into cellular components by mechanical disruption of the tissue into a saline solution. The cell suspensions were centrifuged at 1500 rpm for 10 min. and were exposed to ACK lysis buffer for 1-2 minutes to remove contaminating erythrocytes. ACK lysis buffer consisted of 8.024 mg NH₄Cl, 1.0 mg KHCO₃, 3.722 mg Na₂EDTA.2H₂O in a 1 liter solution of deionized H₂O adjusted to a pH of 7.4. The solution was neutralized with serum containing medium and pelleted. Cells were resuspended in a blocking solution containing 0.5% BSA and antibodies to the Fc receptor CD16/32. This cellular preparation was incubated with specific primary antibody combinations for 30 minutes at 4° C. After incubations, the cells were pelleted and resuspended in buffer and analyzed using a flow cytometer (FACSCalibur, Becton Dickinson). For the isolation of CD4+ CD25+ splenocytes, we used MACS protocols per vendor's instructions to enrich splenocytes of regulatory T cells.

Enzyme-Linked Immunosorbant Assays (ELISAs). Peripheral blood was collected from animals by cardiac puncture and centrifuged at 1500 rpm for 15 minutes to collect serum. Serum was analyzed for IFN-γ and interleukin IL-10 by ELISA. Mouse IFN-γ capture antibody (BD Bioscences) diluted at 2 μg/ml in carbonate buffer (pH 9.0) was physisorbed on 96 well plates at 4° C. overnight. Plates were washed with PBS with 0.1% Triton X-100 (Sigma) and blocked with borate-buffered saline (pH 8.0)/2% bovine serum albumin (Sigma) at room temperature for 2 hours. Standards of mouse recombinant IFN-γ (R&D Systems) and samples were loaded and incubated at 4° C. overnight. Plates were washed and incubated with biotin anti-IFN-γ (1 μg/ml; BD Biosciences) at room temperature for 45 minutes. Plates were washed and incubated with horseradish peroxidase conjugated to avidin (1:1000 in BBS/2% BSA; BD Bioscences) for 45 minutes. Plates were washed and incubated with citrate buffer supplemented with ABTS (Sigma) and 30% hydrogen peroxide, colorized and read at 415 nm on a microplate reader. ELISA for mouse IL-10 (BD OptiEIA IL-10 kit) was performed per vendor's instructions. Each animal's serum was tested in triplicate and data are representative of 4 animals per group for each cytokine analyzed.

Histology. Tissue from the distal ileum, pancreas and liver was harvested from animal groups, one week after treatments. Tissue was fixed in 10% buffered formalin, embedded in paraffin, sectioned to 6-μm thickness, and stained with hematoxylin and eosin. Images are representative of 6 animals per group for the distal ileum and 4 animals for each other tissue.

Immunohistochemistry. Tissues of interest were harvested and placed in a solution of 4% paraformaldehyde and 10% sucrose for 3 hours. Samples were then transferred to a 30% sucrose solution and left overnight to allow for full penetration of the cryoprotectant. Tissues were then embedded in OCT, frozen, and sectioned. Eight-micron thick sections of fixed tissue were washed 3 times in PBS for 15 minutes and blocked with a buffer containing 5% donkey serum and 0.1% Triton X-100 for 30 minutes at room temperature. Slides were washed again with blocking buffer and then incubated with a primary anti-eGFP antibody (Molecular Probes, clone 3EH) at a 1:250 dilution overnight at 4 degrees. After washing with PBS, a secondary donkey anti-rabbit antibody conjugated to Cy3 at a 1:500 dilution in blocking buffer for 30 minutes at room temperature was used for detection. Note that detection of eGFP using this method of indirect immunofluorescence with the stated secondary antibody results in a red signal rather than a green signal, which was better for visualization purposes. The sections were then washed 3 times with blocking buffer and PBS and developed using 3,3′-diaminobenzidine. All histology images were captured on a Nikon Eclipse E800 upright microscope.

Digital Cell Cluster Quantification. Quantification of cell clusters in stained sections was performed on 5 random 40× images per section where at least one cluster was found in that section. Ten sections were made for each tissue from each animal and 3 animals were used in cell trafficking studies. Clusters were visually distinct and defined as a local aggregate of at least 10 eGFP+ cells.

Statistical Analysis. For flow cytometry data, median values±standard deviations are reported. For cytokine analysis, results were analyzed using an unpaired Student's t-test given an unskewed data set and assuming a normal distribution. Significance values of P<0.05 were considered statistically significant. Results are given as a mean±standard error of the mean.

Results

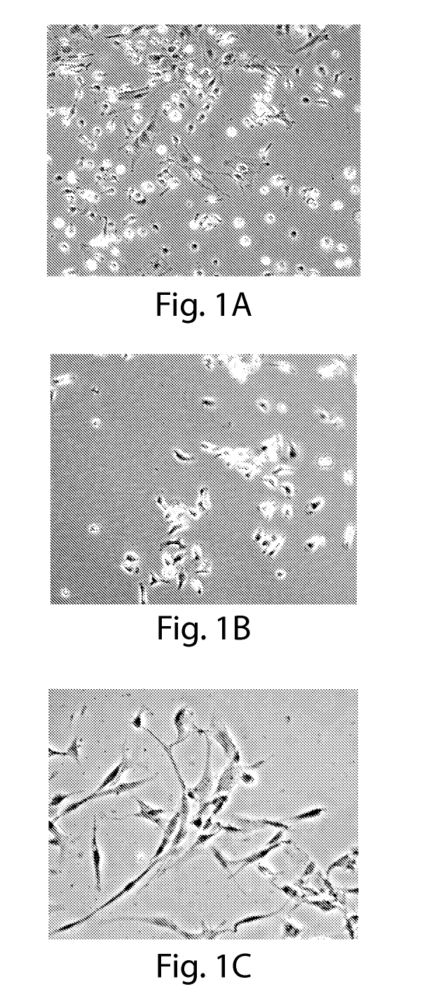

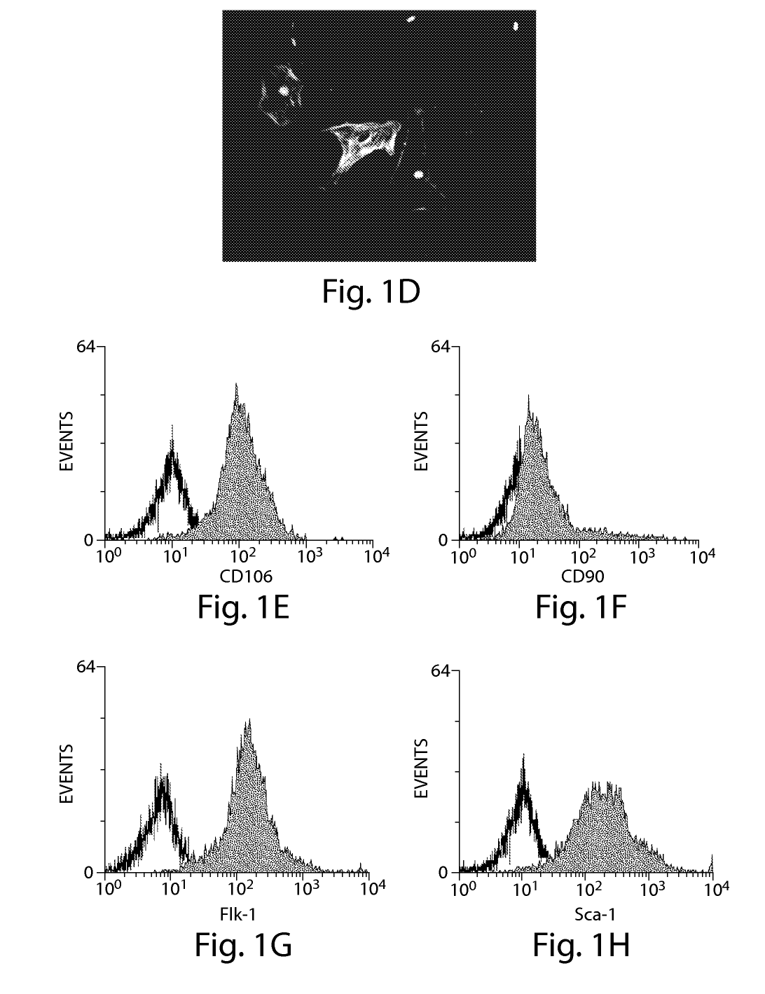

Purification and Characterization of Marrow Stromal Subpopulation. The anchorage-dependent population of bone marrow cells, also known as stromal cells, consists of a relatively heterogenous mixture of cells (FIG. 1A). Evidence exists that a subpopulation of this heterogeneous mixture, which displays phenotypic similarities to MSCs, is responsible for the immunosuppressive effect of the marrow stroma. For the current study, we purified this subpopulation by immunodepleting adherent bone marrow cells of CD11b+ and CD45+ cells using MACS. The CD11b+ CD45+ cell fraction resembled macrophage-like cells (FIG. 1B), while the negative fraction had a more fibroblastoid morphology (FIG. 1C). These fibroblastoid cells were found to be positive for α-smooth muscle actin expression by immunocytochemistry (FIG. 1D). The immunophenotype of the CD11b− CD45− fraction was CD106+, Flk-1+, Sca-1+ and CD90− as determined by cytofluorimetry (FIGS. 1E-H). The phenotype of these cells is identical to C57Bl/6 MSCs (Peister, A. et al., Blood 103, 1662-8 (2004); Baddoo, M. et al., J Cell Biochem 89, 1235-49 (2003)) and will be referred to as MSCs herein for the sake of convenience.

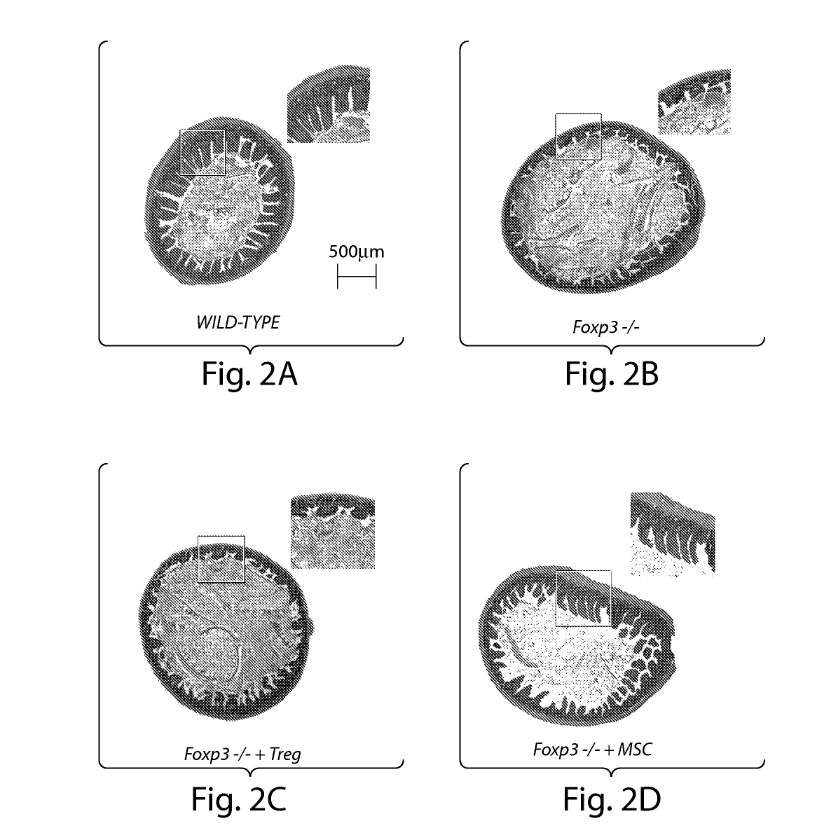

MSCs Suppress Histopathological Changes of Target Organs in Foxp3^{sf} Mice. We evaluated the immunosuppressive efficacy of different cell-based transplantation strategies in Foxp3^{sf} mice, which have widespread autoimmunity due to inefficient peripheral tolerance. Formulations using MSCs were compared with Tregs—the suppressor cells that are deficient in Foxp3^{sf} mice because of the genetic mutation. At 4 weeks of age, Foxp3^{sf} mice were infused with 3×10⁵ cells MSCs intraperitoneally (i.p.) and were sacrificed 1 week post-infusion. For comparison, we used age- and sex-matched mice of the following groups: (a) wild-type C57Bl/6, (b) Foxp3^{sf} treated with vehicle, and (c) Foxp3^{sf} mice treated with MACS-selected CD4+ CD25+ T lymphocytes (3×10⁵ cells)—a T_{reg} phenotype, and hence the most stringent control. Self-reactive T cells can be found in many target organs of Foxp3^{sf} animals, such as skin, endocrine glands and the GI tract. We harvested the distal ileum, pancreas and liver, as representative tissue targets of autoimmunity and examined histopathology. The most dramatic histological change was found in the distal ileum. Foxp3^{sf} animals treated with MSCs regenerated crypt structures similar to wild-type while untreated and Tregs-treated animals failed to do so (FIGS. 2A-D). We observed this GI finding in 4 of 6 animals tested, whereas no vehicle treated mice and only one T_{reg}-treated mouse showed any signs of regrowth.

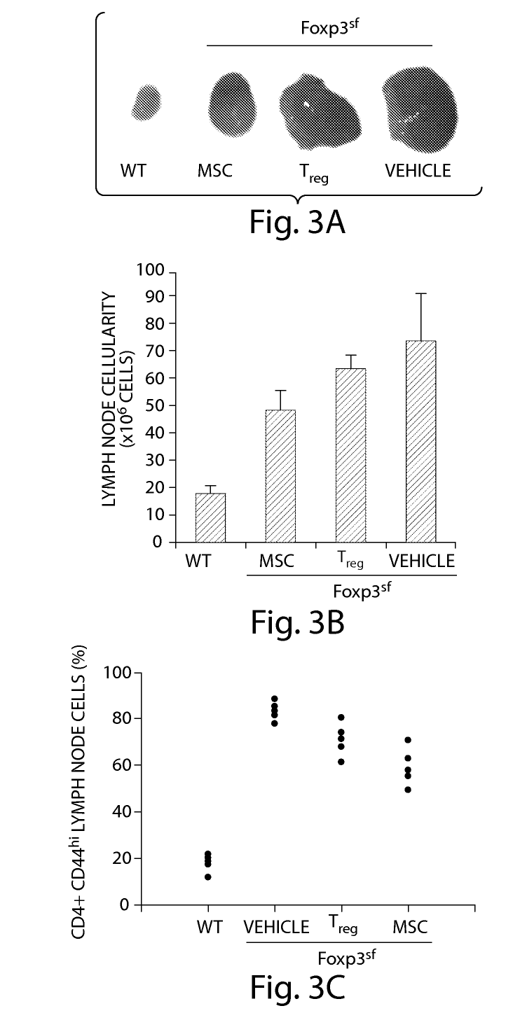

Reduction of Cellularity and Activated T Cells in Mesenteric Lymph Nodes of Foxp3^{sf} Mice after MSC Infusion. We then analyzed lymphoid tissue associated with the intestine for changes in disease. Mesenteric lymph nodes (MLNs) are typically enlarged when there is adjacent inflammation of intestinal tissue. We harvested MLNs and measured total cellularity at 7 days post-infusion of cell transplants. Representative gross histology for MLNs within each group is shown in FIG. 3A. Draining MLNs remained hypercellular in Foxp3^{sf} mice when treated with vehicle (73.5±8.1×10⁶ cells) were compared to wild-type (17.8±2.8×10⁶ cells), whereas cellularity was reduced by MSC (48.1±7.3×10⁶ cells) or Tregs treatment (63.2±5.3×10⁶ cells) when compared to mutant mice (FIG. 3B). Lymph node cells were isolated, gated for CD4 expression, and analyzed for the cell surface activation marker, CD44 using flow cytometry. Compared to wild-type mice (18.8%±3.8), the majority of CD4+ lymph node cells were activated in mutant mice (83.4%±4.0; FIG. 3C). The CD44^{hi} population was reduced in both MSC and T_{reg} treatments (57.9%±8.1 versus 69.2±7.0, respectively). Overall, treatment with MSCs was qualitatively more remarkable in effect than with T_{regs} with respect to suppressing local inflammation in the MLN.

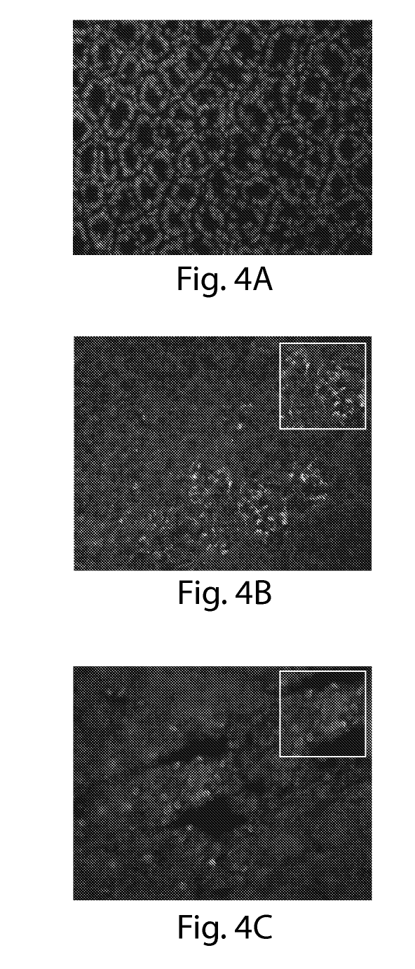

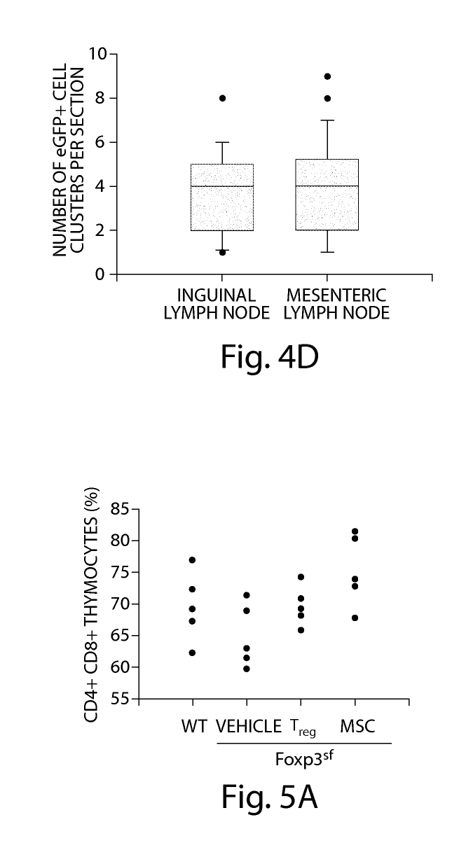

MSCs do not Engraft in Intestine, but Rather Ancillary and Gut-Associated Lymph Nodes. After a tissue- and cell-specific effect of MSC treatment was observed, we attempted to delineate whether this therapy was due to MSC-mediated regeneration of gut tissue versus an alteration of the immunological attack at the intestine. We infused 3×10⁵ eGFP-labeled MSCs i.p. and harvested the distal ileum at 7 days post-treatment. We found no appreciable eGFP+ cells in the intestinal tissue at the 7-day time point (FIG. 4A). On the contrary, eGFP+ cells were detected in the MLN at a significant proportion of the graft relative to intestinal tissue (FIG. 4B). Clusters of eGFP+ cells were found in a network with each cell having a distinct fibroblastoid morphology (FIG. 4B inset). To determine if this engraftment was specific to MLNs in this model of autoimmune disease, we harvested inguinal lymph nodes as an ancillary site. Transplanted cells were also found in inguinal nodes (FIG. 4C). Semi-quantitative image analysis of lymph node engraftment, as assessed by the number of clusters counted, showed no differences between the two sites (FIG. 4D). However, the majority of the cells found within inguinal lymph nodes were not fibroblastoid in morphology and did not form a network.

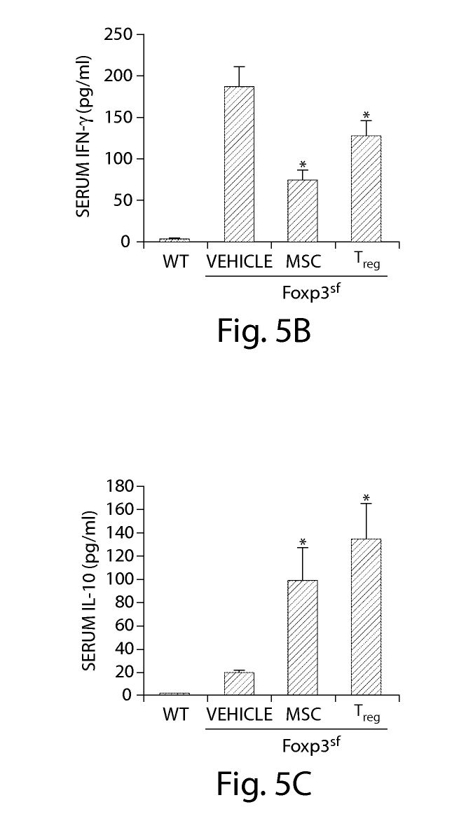

Systemic Evidence of Immunosuppression in Foxp3^{sf} Mice after MSC Treatment. Since we had observed engraftment of MSCs in lymph nodes in two anatomically remote sites, we hypothesized that the cell transplant may have had systemic immunological effects. Two circumstantial measures of systemic immunsuppression are: (1) an increase in the percentage of newly formed CD4+CD8+ thymocytes; and (2) a change in the serum cytokine profile. After cell treatment we observed no difference in total thymocyte number (data not shown), but found that the number of CD4+CD8+ thymocytes of animals treated with MSCs (74.0%±5.6) were increased relative to wild-type (69.2%±5.5), untreated (63.0%±7.6), and Tregs treated (69.2%±3.1) animals (FIG. 5A). In addition, serum IFN-γ (FIG. 5B), an indicator of cell-mediated immune responses and IL-10 (FIG. 5C), a potent immunosuppressive cytokine, were shifted in favor of a global downregulation of the immune system after MSC treatment.

Discussion

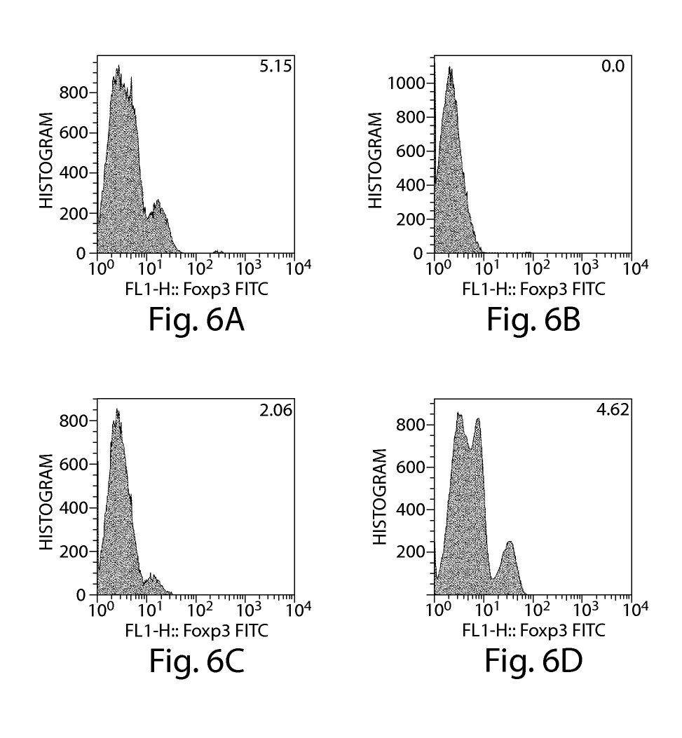

The bone marrow stroma has been identified as a unique site of regenerative and immunosuppressive cells. Many studies have reported inhibition of T lymphocyte functions when cocultured with MSCs by cell contact-dependent and independent mechanisms. However, the use of MSCs as a cellular therapeutic for autoimmune diseases has not been fully explored. We chose to stringently test the efficacy of MSCs as a treatment for autoimmune disease by transplanting these cells in Foxp3^{sf} mice, which lack one mode of peripheral tolerance due to a genetic mutation in the transcription factor Foxp3 that leads to a deficiency in regulatory T cells. Since it has been reported that MSCs can induce the proliferation of CD4+ CD25+ T lymphocytes in vitro (Prevosto, C. et al., Haematologica 92, 881-8 (2007); Aggarwal, S. et al., Blood 105, 1815-22 (2005); Maccario, R. et al., Haematologica 90, 516-25 (2005)), albeit without rigorous analysis of Foxp3 protein expression, the use of Foxp3^{sf} mice as a testbed should not be confounded by such an indirect pathway of therapeutic benefit. As a corollary to our study, we attempted to use our animal model deficient in the T_{regs} pool to test this hypothesis in vivo. In wild-type mice, 5% of splenocytes were reactive to Foxp3 demonstrating a compartment of peripheral splenocytes is devoted to maintaining tolerance (FIG. 6A). Foxp3^{sf} mice treated with MSCs showed no Foxp3 expression in the spleen (FIG. 6B), consistent with the genetic defect in these animals and the finding that MSCs do not express Foxp3. Infusion of 3×10⁵ T_{regs} into mutant animals showed a detectable signal in the splenic T_{regs} pool (FIG. 6C). In contrast, infusion of 2.5×10⁵ T_{regs} and an adjuvant dose of MSCs (2.5×10⁴ cells) resulted in an approximate doubling of the T_{regs} pool (FIG. 6D). This is the first preliminary evidence showing that MSCs can expand the T_{regs} pool in vivo using an animal model deficient in T_{regs} (N=2). More importantly, the current study does not rule out a MSC-T_{reg} interaction in vivo that can amplify the immunosuppressive efficacy of MSC transplantation by secondary effects of boosting endogenous Treg-mediated peripheral tolerance. Our preliminary results suggest this may be a real phenomenon in vivo and we will further explore this phenomenon in Example 2.

We infused MSC transplants in a multi-organ autoimmunity model and screened various tissue targets of autoimmune attack. Since Foxp3^{sf} mice are essentially moribund 4-6 weeks after birth because of the magnitude and nature of their autoimmunity, this study looked at short-term benefits of cell transplantation during active disease. We provide the first evidence that MSCs can specifically ameliorate autoimmune enteropathy. We have shown that MSCs infused into these autoimmune mice led to striking improvements in the distal ileum. The regrowth of villous structures was observed 7 days after MSC treatment, whereas T_{regs} treated animals still had visible disease. There are different potential explanations for ileal regrowth based on experimental evidence. Previous studies have shown that bone marrow-derived mesenchymal cells can give rise to newly-formed myofibroblasts and vasculature in a physiological response to chemically-induced colitis in mice and humans (Andoh, A. et al., J Gastroenterol 40, 1089-99 (2005); Brittan, M. et al., Gastroenterology 128, 1984-95 (2005)). Furthermore, we and others have shown that MSCs can promote regeneration by paracrine stimulation of endogenous self-replicating tissue cells (van Poll, D. et al., Hepatology, in press (2008)) and stem cell populations (Munoz, J. R. et al., Proc Natl Acad Sci USA 102, 18171-6 (2005)). Taken together, our result could have been due to cell homing to the distal ileum and: (a) a direct regenerative response of MSCs, (b) paracrine signals to promote intestinal stem cell expansion and differentiation, or (c) inhibition of immunological attack on the tissue and allowance for natural regeneration of villi. We did not observe any infused eGFP-MSCs in the distal ileum ruling out direct or local, paracrine interactions between MSCs and intestinal cells. The natural replenishment of cells in the GI tract during normal tissue turnover is primarily due to an endogenous stem cell population located in the crypt, which in the mouse can occur in 2-3 days (Cheng, H. et al., Am J Anat 141, 537-61 (1974); Booth, C. et al., J Clin Invest 105, 1493-9 (2000); Bach, S. P. et al., Carcinogenesis 21, 469-76 (2000)). Thus, it is likely that the regeneration of villi was not due to MSC differentiation or paracrine actions, but rather an inhibition of the immune response to the tissue and repopulation of parenchymal cells by crypt stem cells.

To support the concept that MSCs may have modulated the immune reaction at the GI tissue target, we analyzed gut-associated lymph nodes. We observed a decrease in MLN cellularity and activated CD4+ lymph node cells, which support this hypothesis. Inhibition of T cell activation by MSCs may be the result of selective apoptosis of activated T cells, prevention of further T cell activation by direct or indirect (e.g., licensing tolerance-inducing antigen presenting cells (Li, Y.P. et al., J Immunol 180, 1598-608 (2008)) mechanisms or dedifferentiation of activated T cells, although more mechanistic studies are warranted. These results are consistent with the benefit seen with MSCs in severe GVHD, a condition which is thought to matriculate from the Peyer's patches of intestinal tissue (Murai, M. et al., Nat Immunol 4, 154-60 (2003)).

We identified MSCs organized in clusters in engrafted tissues, specifically lymph nodes. Others have also described clusters of engrafted human fetal MSCs that had homed to the bone marrow after in utero treatment of children with osteogenesis imperfecta (Le Blanc et al., J Intern Med 262, 509-25 (2007)), although it was not determined if this a clonal population derived from a single engrafted cell or a local distribution of transplanted cells based on circulatory patterns intrinsic to the tissue of study. Interestingly, there were no relative differences in the number of clusters in MLNs when compared to an anatomically distinct lymph node bed in the inguinal space. Instead, MSCs were morphologically different within the MLN tree displaying spiculated projections and formed a fibroblastoid network. The relevance of this morphological difference is unclear, although we speculate that the engrafted cells seemed more differentiated and integrated into the stroma of the MLN and this may be relevant to MSC functions necessary for therapeutic gains. In the treatment of experimental encephalomyelitis, MSCs were also localized in secondary lymphoid organs including the spleen and lymph nodes (Zappia, E. et al., Blood 106, 1755-61 (2005)), the latter finding of which was reproduced and extended to other lymph nodes in this study. Prior work has shown trafficking of peritoneal infused lymphocytes to MLNs and pancreatic lymph nodes (Turley, S. J. et al., Proc Natl Acad Sci USA 102, 17729-33 (2005)) suggesting these may be likely sites of tolerance induction after MSC transplantation assuming similar homing mechanisms exist in MSCs. Ultimately, these data suggest certain “tropic” aspects of MSC transplantation efficacy that may hinge upon endogenous properties of the host, namely: (a) a tissue-associated lymphoid bed for engraftment and immunomodulation, and (b) a host-autonomous system of regeneration to restore tissue function and homeostasis.

Moreover, we observed an increase in the number of CD4+ CD8+ thymocytes and changes in the serum cytokine profile after MSC infusion. T cells are born in the thymus and studies have shown that the mesenchyme is integral in the proper education of T cells (Anderson, G. et al., Nat Rev Immunol 1, 31-40 (2001)). In addition, other investigators have shown that infused MSCs can migrate and functionally engraft at lower numbers in the thymus (Liechty, K. W. et al., Nat Med 6, 1282-6 (2000)). Our study only examined engraftment in lymph node tissue, so a more comprehensive view of MSC engraftment in this, and other, autoimmunity models may elucidate the role of transplanted cells on thymic cell output and function. We have shown that human MSCs secrete immunomodulatory molecules that can provide a significant survival benefit to rats undergoing acute liver failure (Parekkadan, B. et al., PLoS ONE 2, e941 (2007)) and also shift the serum cytokine profile (van Poll, D. et al., Hepatology, in press (2008)) independent of cell contact mechanisms. We infer the change in the cytokine profile in the study may be a paracrine effect of engrafted or lysed infused cells that resulted in global serological differences, although there are obvious pathological and pharmacological differences between the former study and the current one to consider. More importantly, these studies may indicate a systemic immunosuppressive effect after MSC transplantation. It may be argued that successful treatment of autoimmunity may require a systemic-scale approach and this study validates MSC therapy within such a context.

In conclusion, we report the first use of MSCs in a multi-organ model of autoimmunity, including a MSC-specific amelioration of autoimmune enteropathy.

### Example 2

Materials and Methods

Mice. C57Bl/6 mice between 4 to 6 weeks of age were purchased from Charles River Laboratory. Animals were maintained in a light-controlled room (12-h light-dark cycle) at an ambient temperature of 25° C. with chow diet and water ad libitum. The animals were cared for in accordance with the guidelines set forth by the Committee on Laboratory Resources, National Institutes of Health. All experimental procedures performed were approved by Subcommittee on Research Animal Care and Laboratory Animal Resources of Massachusetts General Hospital.

Cell Culture. Syngenic MSCs were kindly donated by the Center for Gene Therapy at Tulane University. MSC expansion medium consisted of alpha-MEM without deoxyribonucleosides and ribonucleosides (Gibco), 10% lot selected FBS (Atlanta Biologicals), 100 U/ml penicillin (Sigma), and 100 μg/ml streptomycin (Sigma). NIH 3T3-J2 fibroblasts were a kind gift from Dr. Howard Green and cultured according to donor's protocol.

Mesenchymal Stem Cell Conditioned Medium (MSC-CM). For the generation of MSC-CM, cells were allowed to grow to 70-80% confluence, washed thoroughly and cultured in 15 ml serum free Dulbecco's Modified Eagle's Medium supplemented with 0.05% bovine serum albumin. Conditioned medium was collected 24 hours later and concentrated 25-fold using ultrafiltration units (Millipore, Bedford, Mass.) with a 3 kD cutoff The conditioned medium from an equivalent of 1×10⁶ cells was concentrated to 100 μl for intravenous use in mice.

Colitis Induction and Cell Transplantation. C57Bl/6 mice (male, 6-8 weeks) were weighed, fasted for 24 hours, and re-weighed to document baseline data. Mice were then anesthesized using a 300 uL i.p. injection of 2.5% Avertin (40× stock: 1 g/mL of tribomoethyl alcohol solubilized in tertiary amyl alcohol; Sigma). Prior to IBD induction, MSCs or MSC conditioned medium were infused by tail vein injection or i.p. at different cell doses. Fibroblast infusion and saline infusions will serve as controls. To induce colitis, mice were administered 100 uL of a haptenating agent, trinitrobenzosulfonic acid (TNBS), at a 1:1 ratio of 5 mg/mL of TNBS to 100% ethanol (used to disrupt the epithelial barrier) per rectum. The mixture was slowly administered into the lumen of the colon via a 22 g catheter (Becton Dickinson) fitted onto a 1-mL syringe with the animals under Avertin anesthesia, and mice were then kept in a vertical position for 30 seconds. Control mice received 50% ethanol in phosphate-buffered saline (PBS) using the same technique as previously described. After induction, mice were observed for weight changes and mortality on a daily basis. In therapeutic trials, cells were infused two days after TNBS induction and similar physical parameters were measured.

Test for Fecal Occult Blood. Fresh feces from animals was procured three days after TNBS induction and tested for fecal occult blood per vendor's instructions (Hemoccult Sensa, Beckman Coulter). The tests were read by an independent observer and given a semi-quantitative score of 0-5 as shown by the color indicators provided by the manufacturer.

Isolation and Analysis of Lymph Node Cells. Lymph nodes were dissected from experimental mice and dissociated into cellular components by mechanical disruption of the tissue into a saline solution. The cell suspensions were centrifuged at 1500 rpm for 10 minutes and were exposed to ACK lysis buffer for 1-2 minutes to remove contaminating erythrocytes. The solution was neutralized with serum containing medium and pelleted. Cells were resuspended in a blocking solution containing 0.5% BSA and antibodies to the to Fc receptor CD16/32. This cellular preparation was incubated with anti-Foxp3-FITC (eBiosciences) and analyzed using flow cytometry.

Gross and Microscopic Histology. Lymph nodes and intestinal tissue from animal groups were harvested one week after treatments. Lymph nodes and the colon, dissected from the ileocecal junction to the sigmoid rectum, were prepared for gross histological imaging using a digital camera and subsequently prepared for microscopic evaluation. Intestinal tissue was fixed in 10% buffered formalin, embedded in paraffin, sectioned to 6-μm thickness, and stained with hematoylin and eosin.

Results

MSC Transplant, and not MSC-CM, Inhibits Physical Evidence of TNBS-Induced Colitis. We have previously demonstrated that: (a) human MSC secreted factors can reverse hepatotoxin-induced fulminant hepatic failure and (b) mouse MSC transplants can inhibit autoimmune enteropathy without a need for regulatory T cells. In this study, we sought to test the efficacy of these treatments (i.e., MSC molecules vs. MSC transplant) in a more classical model of colitis. One such model that leads to a T_{H}1 immune response resembling CD can be induced by administering TNBS solubilized in ethanol (disrupts epithelial barrier) directly into the colon. TNBS is a hapten that binds to endogenous proteins and forms neo-antigens which are the target for autoimmune attack. In a prevention trial, animals were randomized and treated with saline (internal control), or unit doses of NIH-3T3 fibroblasts (cell control) or mMSCs. Animals then were anesthesized and administered a 1:1 chemical mixture per rectum consisting of either ethanol:saline (sham control) or ethanol:TNBS.

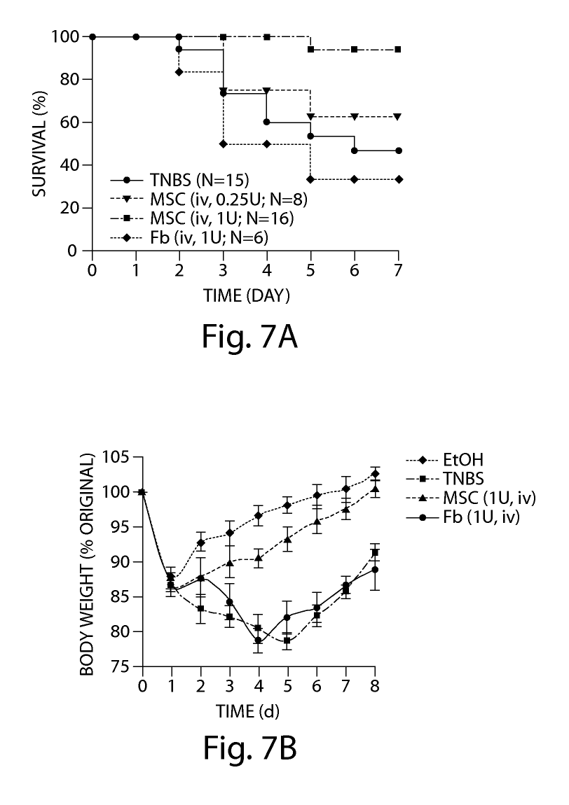

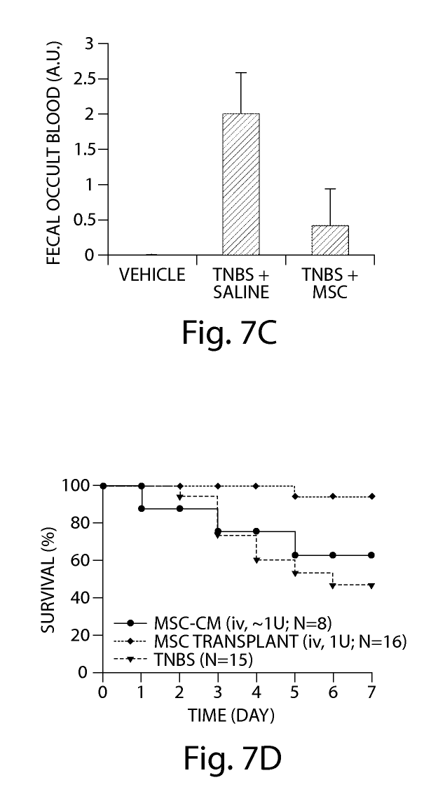

Intravenous treatment with 1 U of MSC improved all physical evidence of colitis after MSC transplantation. A significant survival benefit was observed, with 94% of animals surviving after TNBS induction when treated with MSCs, compared to 47% and 33% when treated with vehicle or a fibroblast control, respectively (FIG. 7A). Approximately 63% of mice survived after infusion of 0.25 U of MSCs, which was not statistically significant but suggested survival was a function of MSC dose. No significant improvement in survival was seen when MSCs were infused intraperiotoneally (FIG. 7B) compared to controls. In addition, we measured animal weight and fecal occult blood as indicators of disease. A maximal 22% reduction in body weight loss was observed post-fast in animals treated with vehicle or 1 U of fibroblasts i.v. with a final 14% loss at the study endpoint (FIG. 7C). MSC-treated animals had a maximal 13% loss of body weight post-fast with a 1% gain in body weight at the study endpoint. Similarly, stool guiac tests performed on day 3 post-induction showed nearly complete absence of occult blood in feces of MSC treated mice when compared to vehicle treatment (FIG. 7D). Finally, MSC-CM infused prior to induction of disease did not provide an equivalent survival benefit to mice suggesting that secreted factors alone are not responsible for the therapeutic efficacy of an MSC transplant in colitis (FIG. 7E).

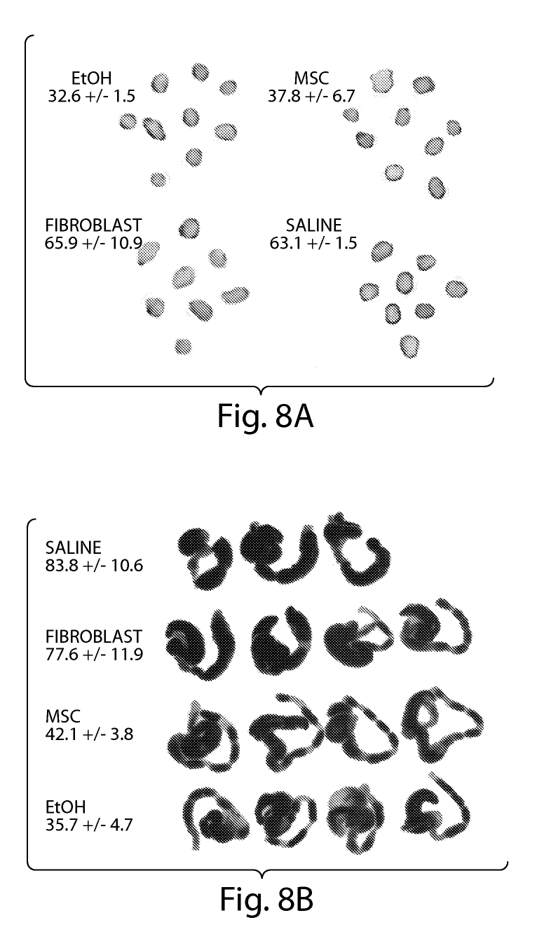

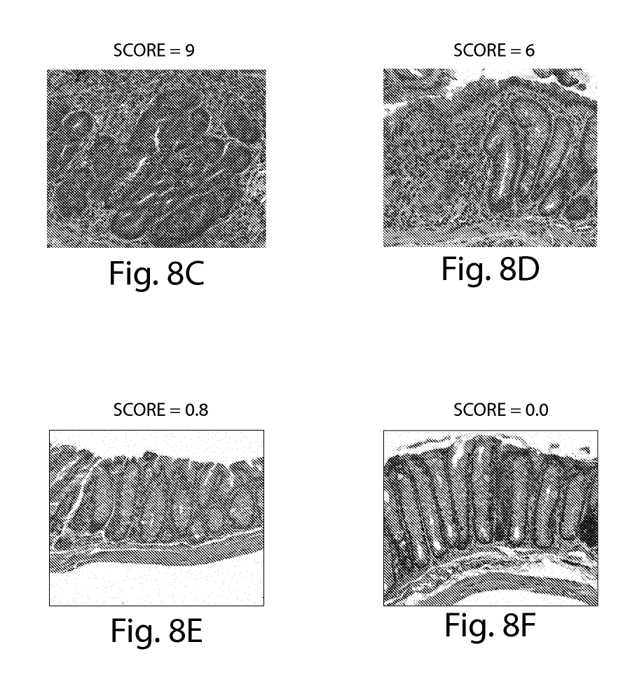

Prevention of Histopathological Changes in Target Organs by MSCs. At the study endpoint, we examined histopathological changes in the colon and associated lymph nodes of the surviving animals. Vehicle treated animals had macroscopically enlarged lymph nodes and thickened colonic walls (FIG. 8A), which is likely due to edematous fluid, with major lesions and ulcerations found in distal half of the colon. On the other hand, mice transplanted with MSCs showed no gross signs of disease and were qualitatively similar to mice that had minor inflammation caused by the local ethanol infusion (FIGS. 8B, C). Mesenteric lymph node cellularity and colonic weight/length ratio were quantified and listed in Table 1. Colonic tissue was microscopically examined using conventional H&E methods. Mice treated with MSCs had few, if any, inflammatory infiltrates, crypt abscesses, goblet cell thickening, and loss of tissue architecture when compared to other treated groups (FIGS. 8D-F). Pathological scoring of the tissue quantitatively confirmed the significant differences in histology (Table 2).

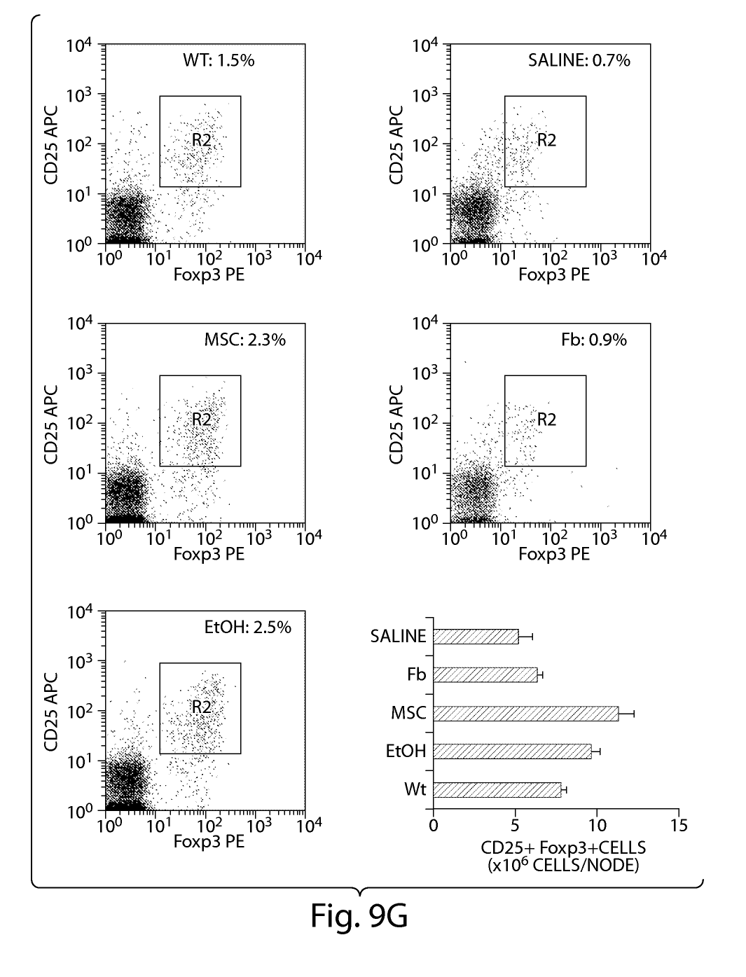

MSC Transplantation Leads to Higher Numbers of Foxp3+ Cells in Mesenteric Lymph Nodes. Based on our prior observation in Example 1 of regulatory T cell (T_{reg}) expansion after co-transplant of T_{regs} and MSCs in T_{reg}-deficient mice, we enumerated the T_{reg} cell number in colitis-induced animals at the study endpoint. Indeed, we saw a preservation of T_{reg} frequency in the lymph nodes of MSC-treated mice consistent with our hypothesis that MSCs may “amplify” their immunosuppression by indirectly expanding endogenous suppressor T cells (FIG. 9A). When infused with 1 U of MSCs, 2.6% of lymph node cells from TNBS-induced mice were Foxp3+ compared to 0.6% and 0.9% in saline and mock cell treated animals, respectively. Moreover, infusion of MSC-CM slightly increased Foxp3+ cells to 1.2% but this was not statistically significant. We further quantified the absolute number of T_{regs} given that there were quantifiable differences in lymph node cellularity (FIG. 9B). Treatment with MSCs had an approximately 2.5 fold absolute number of Foxp3+ cells (9.8±1.7×10⁶ cells) when compared to saline treated animals (3.7±0.5×10⁶ cells).

TABLE 1
Quantitative Histological Analysis of Colitis-Induced Mice

| | Mesenteric Lymph | Colonic |
|---|---|---|
| | Node Cellularity | Weight/Length |
| Treatment Arm | (×10⁶ cells/node) | Ratio (mg/cm) |
| Ethanol | 32.59 ± 1.53 | 35.69 ± 4.72 |
| TNBS | 63.07 ± 1.53 | 83.82 ± 10.66 |
| MSC-CM | 53.82 ± 2.83 | 67.24 ± 7.21 |
| Fibroblast (iv, 1U) | 65.93 ± 10.92 | 77.55 ± 11.93 |
| MSC (iv, 1U) | 37.84 ± 6.67 | 42.10 ± 3.86 |

NOTE:
1U = 1 × 10⁶ cells

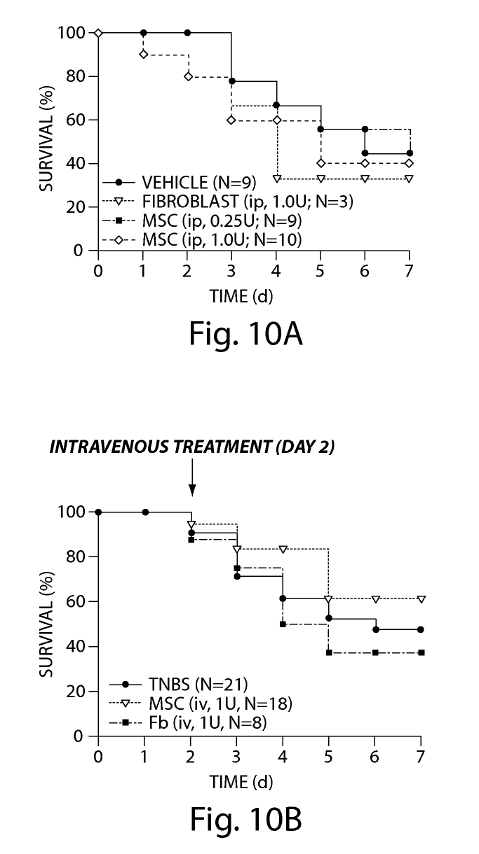

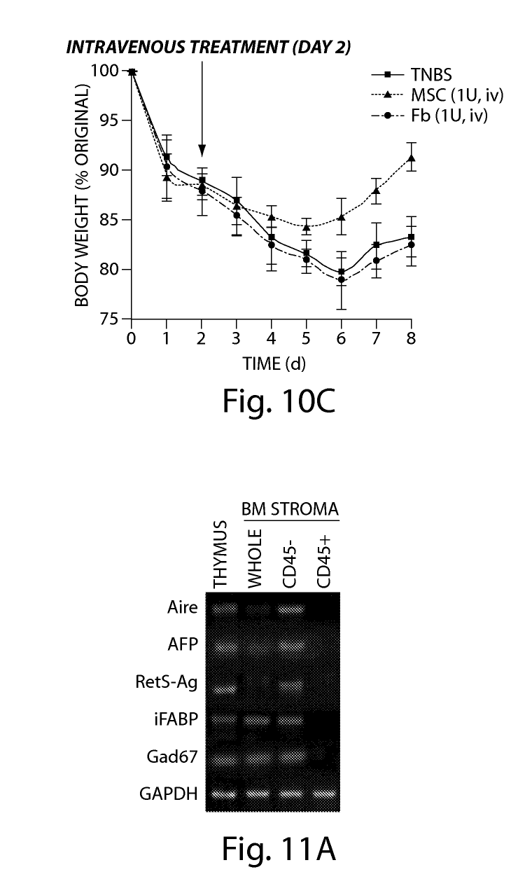

Amelioration of Colitis by MSC Graft After Disease Onset. After identifying an optimal cell dose and analyzing the histopathological response to MSC transplantation in a preventative setting, we tested MSCs therapy after disease onset, which is a more clinically relevant case. We chose to infuse cells two days after TNBS induction, based on our prevention trial which showed that vehicle treated animals began to die at this time point. Intravenous infusion of 1 U of MSCs led to 80% of animals surviving, which was still significant compared to controls (FIG. 10A). In addition, the body weights of MSC treated animals showed only 9% loss in body weight at the study endpoint compared to a 14% loss in control treatments (FIG. 10B).

TABLE 2
Pathological Analysis of Microscopic Colon Sections

| | Average Pathology Score |
|---|---|
| Treatment Arm | (N = 4 per group, max 18) |
| Ethanol | 0 |
| TNBS | 9 |
| MSC (iv, 1U) | 0.8 |
| Fibroblast (iv, 1U) | 6 |
| MSC-CM | 2 |

Discussion

In Example 1, we demonstrated a site-specific benefit in the intestine after MSC transplantation in a multi-organ autoimmunity model. In this study, we further elaborated on these results using a chemically-induced model of colitis. MSC transplantation was found to increase survival prior to and after the onset of disease. Interestingly, intravenous treatment led to a significant survival benefit, distinct from the c intraperitoneal treatment of Foxp3^{sf} mice in Example 1.

Our previous studies demonstrated that MSCs can secrete bioactive molecules that can modulate the inflammatory reaction to liver injury. We did not see any significant benefit to mice treated with MSC-CM prior to colitis induction suggesting that secreted molecules alone did not infer any therapeutic value in this model of colitis. However, we cannot rule out a paracrine effect of MSC therapy because of issues such as the species source of MSC-CM, time of delivery, and the challenge of studying paracrine effects in situ.

MSCs have been shown to generate suppressor cells of both the CD4+ and CD830 lineages in vitro (Prevosto, C. et al., Haematologica 92, 881-8 (2007); Aggarwal, S. et al., Blood 105, 1815-22 (2005); Maccario, R. et al., Haematologica 90, 516-25 (2005)), however few reports exist of this phenomenon in vivo. We had previously shown preliminary evidence in Example 1 that cotransplantation of MSCs and T_{regs} at a 1:10 cell ratio leads to an increase in the number of engrafted T_{regs} in the spleen. In this study, we provide definitive proof that infusion of MSCs can maintain levels of T_{regs} during disease. The maintenance of T_{reg} number could be due to: (a) an increased production of cells from naïve T cells; (b) decreased elimination of T_{regs}; (c) increased proliferation of existing T_{regs}; and/or (d) alterations in trafficking of T_{regs} to the local area. Indeed, many of the secreted factors such as prostaglandin E2 (Aggarwal, S. et al., Blood 105, 1815-22 (2005)) and nitric oxide (Ren, G. et al., Cell Stem Cell 2, 141-50 (2008); Sato, K. et al., Blood 109, 228-34 (2007)) that MSCs produce and that have been shown to be involved in T cell suppression also act as mitogens for regulatory T cell conversion of peripheral naïve T cells (Baratelli, F. et al., J Immunol 175, 1483-90 (2005); Niedbala, W. et al., Proc Natl Acad Sci USA 104, 15478-83 (2007)). In addition, a study had shown that MSCs do not only inhibit the proliferation of T cells, but promote the survival of T cells in a quiescent state under apoptotic conditions (Benvenuto, F. et al., Stem Cells 25, 1753-60 (2007)). Although the details of the mechanism were poorly understood, it is likely that a similar protection of T cell death may also affect regulatory T cells. Moreover, peripheral T_{regs} have a high turnover rate and a somewhat terminally differentiated phenotype suggesting that they may be the remnants of previously activated T cells (Akbar, A. N. et al., Nat Rev Immunol 7, 231-7 (2007)). It is possible that MSCs may enhance this differentiation process before or after T cells have already undergone activation by inflammatory stimuli.

In conclusion, we definitively show that MSC transplantation can be an effective means to prevent and treat colitis in mice. This treatment correlated with a higher local regulatory T cell number in gut-associated lymph nodes indicating that the immunosuppressive signature of MSC transplantation may be amplified through the maintenance of endogenous suppressor cells in vivo.

### Example 3

Materials and Methods

Mice. C57Bl/6 mice between 4 to 6 weeks of age were purchased from Charles River Laboratory. The animals were cared for in accordance with the guidelines set forth by the Committee on Laboratory Resources, National Institutes of Health. All experimental procedures performed were approved by Subcommittee on Research Animal Care and Laboratory Animal Resources of Massachusetts General Hospital. Animals were maintained in a light-controlled room (12-h light-dark cycle) at an ambient temperature of 25° C. with chow diet and water ad libitum.

Antibody and Reagents. The following antibodies were used for flow cytometry and immunochemistry: UEA-1-FITC (Vector Laboratories), biotinylated Ulex europas agglutinin (UEA)-1, biotinylated CD45 (eBiosciences), PD-L1 (Pharmingen), and H-2D^{b} (Pharmingen). Streptavidin microbeads, CD45 and CD11b microbeads along with magnetic columns were purchased from Milenyi Biotec. For immunocytochemistry, anti-mouse AFP was purchased from Santa Cruz Biotechnology.

Isolation and Culture of Bone Marrow-Derived MSCs. Bone marrow was harvested from wild-type and iFABP-tOVA mice after euthanization. Tibias and femurs were dissected and the marrow space was flushed with MSC expansion medium using a 23 gauge needle. Bone marrow plugs were collected on ice, dissociated by repeated passage through an 18 gauge needle and passed through a 70 um filter to remove bony spicules and debris. Approximately 50×10⁶ bone marrow cells were plated on a 100 mm² tissue culture dish and cultured for 3 days to allow for differential adhesion of stromal cells. Non-adherent cells were aspirated on day 3 and the adherent population was cultured in MSC expansion medium for a subsequent 4-10 days to achieve the maximal number of colony forming unit-fibroblast prior to initial passage. Cells were passaged using 0.1% trypsin/0.1% EDTA, and subcultured at a density of 5×10³ cells/cm². All cultures were used between passages 2-8. Prior to use, stromal cells were then depleted of CD11b and CD45 cells using magnetic activated cell sorting (MACS) per vendor's instructions. Long term cultured MSCs were kindly donated by the Center for Gene Therapy at Tulane University and grown in MSC expansion medium. MSC expansion medium consisted of alpha-MEM without deoxyribonucleosides and ribonucleosides (Gibco), 10% lot selected FBS (Atlanta Biologicals), 100 U/ml penicillin (Sigma), and 100 μg/ml streptomycin (Sigma).

Total RNA Isolation and Endpoint/Quantitative RT-PCR. RNA was extracted from 0.1-1.0×10⁶ MSCs using the Nucleospin RNA purification kit (BD Biosciences, Palo Alto, Calif.) per the manufacturer's instructions. Approximately 500-1000 ng of total mRNA was reverse transcribed to cDNA using the Two-Step RT-PCR Kit (Qiagen, Valencia, Calif.) per manufacturer's instructions and amplified in a Perkin Etus Thermal Cycler 480 or a Stratagene Light Cycler. Cycling conditions for PCR were: 1) 50° C. for 30 minutes; 2) 95° C. for 15 minutes ; 3) 40 cycles at 94° C. for 30 seconds, 55° C. for 30 seconds, and 72° C. for 1 minute; and 4) a final extension step at 72° C. for 10 minutes. Primers used for amplification are listed in Table 3. For quantitative analysis, we used a ΔCt method with stated controls with results stated as relative differences between experimental groups. For endpoint analysis, amplified cDNA was run on a 1.2% agarose gel and visualized using a gel imager.

Histology and Immunohistochemistry. The tibia was harvested from wild type animals and fixed, decalcified as previously described (Calvi, L. M. et al., Nature 425, 841-6 (2003)), embedded in paraffin, sectioned to 6-μm thickness, and stained with hematoxylin and eosin. Other paraffin-embedded sections incubated at 60 degrees for 1 hour on a slide warmer and deparaffinized and hydrated through graded levels of xylene clearing followed by ethanol washes. Antigen retrieval was performed by microwaving slides in a sodium citrate buffer (pH 6) for 10 minutes. The endogenous peroxidase was quenched using a 3% H₂0₂/methanol solution for 10 minutes. The slides were then washed 3 times in PBS for 15 minutes and blocked with a buffer containing 5% donkey serum and 0.1% Triton X-100 for 30 minutes at room temperature. Slides were washed again with blocking buffer and then incubated with biotinylated UEA-1 (Vector Laboratories) at a 1:200 dilution overnight at 4 degrees. After washing with PBS, an immunoperoxidase procedure was performed according to vendor's protocols (Vectashield). The sections were then washed 3 times with PBS and counter-stained with methyl green. All histology images were captured on a Nikon Eclipse E800 upright microscope.

TABLE 3
Oligonucleotide Sequences to Study Peripheral Tissue Antigen Expression (SE ID NO:)

| Mouse | | Human | |
|---|---|---|---|
| Primer | Sequence | Primer | Sequence |
| OVA | F: GCTGCAGATCAAGCCAGAGAGC (1) | RetS-Ag | F: CGCAGGGACCTGTACTTCTC (23) |
| | R: ATTGATTTCTGCATGTGCTGC (2) | | R: TCAGGAGAAAGGGGTACGTG (24) |
| RetS-Ag | F: TGACTACCTACCCTGTTCAG (3) | Gad67 | F: AGCACCGCCATAAACTCAAC (25) |
| | R: TTCACTGGATGTGAGCTCTC(4) | | R: ATCTGGTTGCATCCTTGGAG (26) |
| iFABP | F: ACGGCACGTGGAAAGTAGAC(5) | iFABP | F: AAAGAATCAAGCGCTTTTCG (27) |
| | R: AGAAACCTCTCGGACAGCAA (6) | | R: TCCATTGTCTGTCCGTTTGA (28) |
| Il-FABP | F: GGACAGGACTTCACCTGGTC (7) | il-FABP | F: TAATCGAAAAGGCCCACAAC (29) |
| | R: CAAGCCAGCCTCTTGCTTAC (8) | | R: ATGTTGCTTTCCTTGCCAAC (30) |
| CK-8 | F: ATGCTGGAGACCAAATGGAG (9) | CK-8 | F: GACATGGACAGCATCATTGC (31) |
| | R: CCTCATACTGGGCACGAACT (10) | | R: GGCTCTGCAGCTCCTCATAC (32) |
| A33 | F: CCGAAGTCAGACGGAAAGAG (11) | AFP | F: AGCTTGGTGGTGGATGAAAC (33) |
| | R: TGCTGGAGGTGCAGATGTAG (12) | | R: TCTTGCTTCATCGTTTGCAG (34) |
| INS-1 | F: TGTTGGTGCACTTCCTACCC (13) | INS-1 | F: GGGAACGAGGCTTCTTCTAC (35) |
| | R: TAGAGGGAGCAAATGCTGGT (14) | | R: CACAATGCCACGCTTCTG (36) |
| Gad67 | F: TGCAACCTCCTCGAACGCGG (15) | A33 | F: CTTCGCAGGGAAAGAGTGTC (37) |
| | R: CCAGGATCTGCTCCAGAGAC (16) | | R: GACTGCTCAGCATTGTTGGA (38) |
| AFP | F: CTCAGCGAGGAGAAATGGTC (17) | B-actin | F: CTCAGCGAGGAGAAATGGTC (39) |
| | R: CTCAGCGAGGAGAAATGGTC (18) | | R: CTCAGCGAGGAGAAATGGTC (40) |
| Aire | F: TGGTCCCTGAGGACAAGTTC (19) | Aire | F: GAACGGGATTCAGACCATGT (41) |
| | R: TGAATTCCGTTTCCAAGAGG (20) | | R: AACCTGGATGCACTTCTTGG (42) |
| GAPDH | F: ATGACATCAAGAAGGTGGTG (21) | MOG | F: TCACCTGCTTCTTCCGAGAT (43) |
| | R: CATACCAGGAAATGAGCTTG (22) | | R: GAGGAGAACCAGCACTCCAG (44) |

Statistical Analysis. For flow cytometry data, median values±standard deviations are reported. Results were analyzed using an unpaired Student's t-test given an unskewed data set and assuming a normal distribution. Significance values of P<0.05 were considered statistically significant. Results are given as a mean±standard error of the mean.

Results

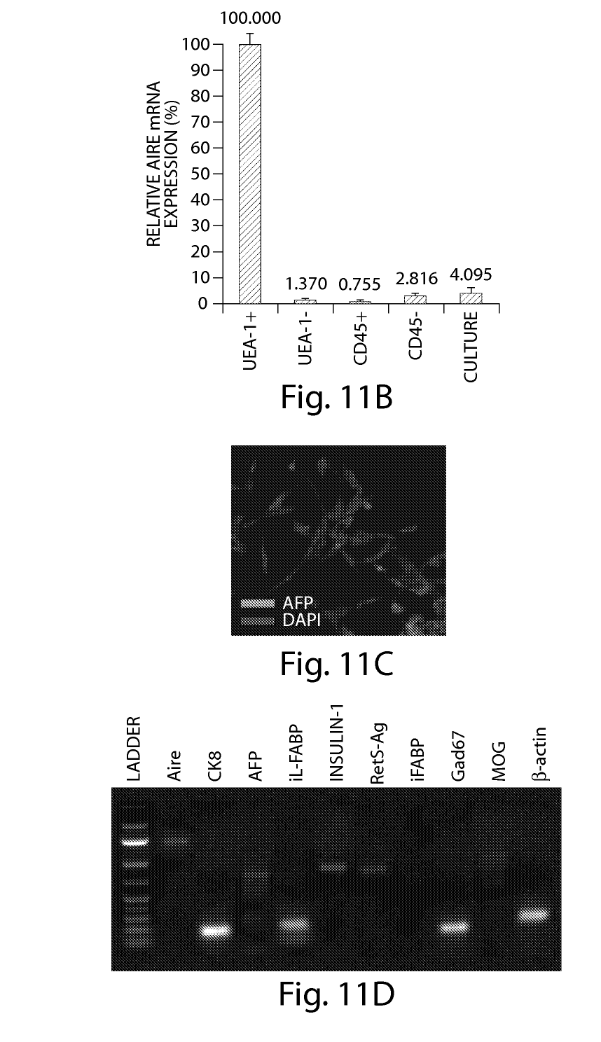

MSCs Express mRNA and Protein for a Variety of Endogenous and Transgenic pTAs after Long-Term Culture Expansion. We hypothesized that MSCs may present pTAs in a similar fashion to other non-hematopoietic cells of lymphoid origin. To determine if mMSCs can present promiscuous antigens, we first analyzed mRNA expression for a panel of pTAs. After 7 days of in vitro culture, gene expression profiling revealed that adherent bone marrow stromal cells expressed all pTAs surveyed mirroring the expression of thymic tissue (FIG. 11A). In addition, when we fractionated the stromal population based on CD45 expression, we found that pTA transcription was restricted to the CD45− population, which is consistent with a MSC phenotype. This expression pattern also included the transcription factor AIRE, which is known to be essential for promiscuous gene expression in mTECs. We then determined protein expression of one pTA, alpha-fetoprotein (AFP), by immunocytochemistry. FIG. 11 B shows that all CD45− cells were reactive to AFP indicating that the message was transcribed into a properly folded protein. More importantly, pTA expression was found in human cells and is retained in long-term culture (FIG. 11C). Based on our prior work in intestinal autoimmune disease, we then quantified the amount of AIRE and intestinal pTAs using quantitative RT-PCR. In all analyzed genes, mouse MSCs expressed approximately 2-7% of the mRNA transcripts for AIRE and pTAs compared to mTECs (FIG. 11D).

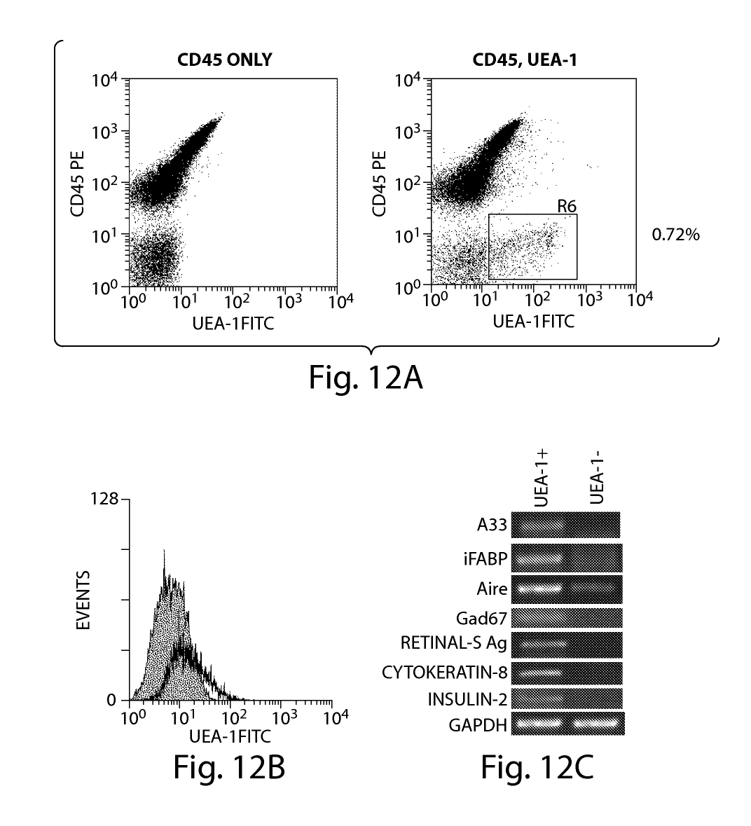

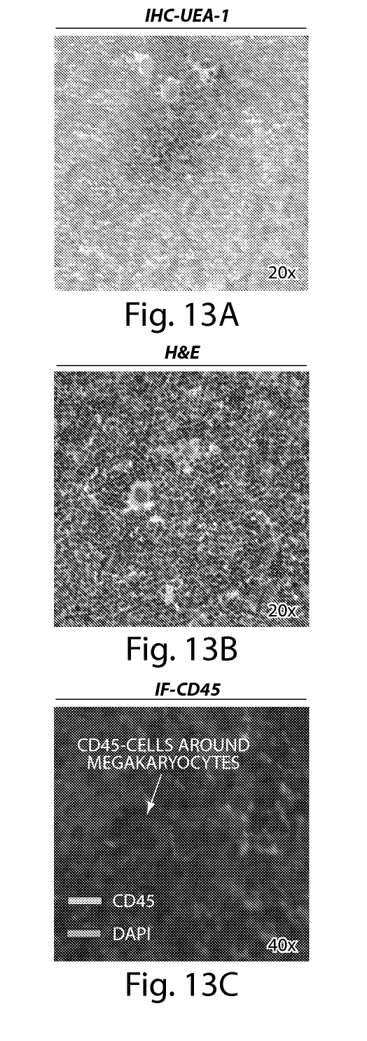

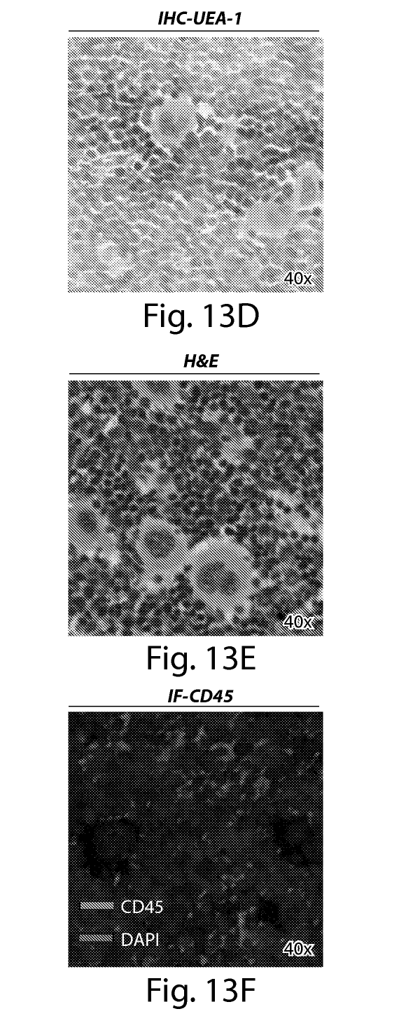

Clusters of UEA-1+ marrow cells Express mRNA for pTAs. Reactivity to UEA-1 has been shown to specifically label mTECs and LNSCs demonstrating a correlation with this lectin to pTA expression. Prior reports state the presence of CD45+ cells with UEA-1 expression in the bone marrow that suggested an endothelial cell or megakaryocyte precursor. We found CD45− UEA-1+ cells from a freshly prepared bone marrow aspirate (FIG. 12A). In addition, we found that UEA-1+ cells specifically expressed pTAs (FIG. 12B). After long-term culture of MSCs, UEA-1 activity was lost (FIG. 12C). We sought to visualize UEA-1+ cells in the bone marrow space. Using an immunoperoxidase method, we saw UEA-1+ cells in perivascular spaces in clusters (FIGS. 13A, D). In addition, these cells were found in close proximity to megakaryocytes. Expression of CD45 was lacking in cells around megakaryocytes relative to other CD45+ cells (FIGS. 13C, F). Due to the high autofluorescence of the bone marrow cavity and the limited amplification of biotinylated molecules, we failed to identify co-immunostaining for CD45 and UEA-1.

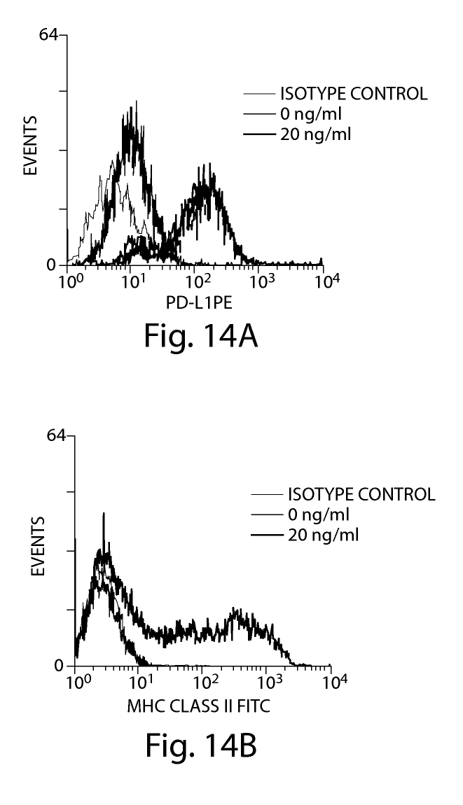

Upregulation of Antigen Presentation and Inhibitory Co-Stimulation Molecules After Cytokine Stimulation. In addition, we examined the ability of MSCs to upregulate antigen presentation and inhibitory co-stimulation molecules after cytokine stimulation. After 24 hours of incubation with interferon (IFN)-γ, a TH1 cytokine secreted by many cell types in response to inflammation, mMSCs showed prominent upregulation of programmed death-ligand 1 (PD-L1) and MHC class II (FIG. 14). These data suggest that mMSCs may actively respond to inflammation by expressing inhibitory surface proteins such as PD-L1, which is known to anergize T cells (Sharpe, A. H. et al., Nat Immunol 8, 239-45 (2007); Barber, D. L. et al., Nature 439, 682-7 (2006)) as well as communicating with CD4+ T cells via class II presentation.

Discussion

The ability of MSCs to present exogenous antigens to T cells has been previously studied by a number of investigators (Krampera, M. et al., Blood 101, 3722-9 (2003); Chan, J. L. et al., Blood 107, 4817-24 (2006); Stagg, J., et al. Blood 107, 2570-7 (2006)). Furthermore, MSCs have been found to inhibit the proliferation, cytotoxicity and number of lymphokine-producing antigen-specific T cells (Krampera, M. et al., Blood 101, 3722-9 (2003)). These results were independent of MSC secreted factors and the function of other antigen presenting cells or regulatory T cells. In these reports, the effect of IFN-γ was observed to upregulate antigen presentation machinery and capability of MSCs to stimulate antigen-specific T cells. IFN-γ stimulation led to the upregulation of PD-L1 and MHC class II. The expression of pTAs was found to be maintained in long-term culture as opposed to mTECs, which rapidly undergo apoptosis upon AIRE expression. We hypothesize that the relatively high level of mRNA of mTECs observed by qRT-PCR analysis may lead to a significant stress on the mTECs ultimately causing programmed cell death or autophagy. Moreover, MSCs may be endowed with robust mRNA and protein synthesis machinery that may allow them to tolerate moderate stresses well.

Recent studies have shed light on how tolerance is induced both centrally and peripherally. In both locations, there exist specialized antigen presenting cells that are essential for preventing autoimmunity. Within the localized compartment of the thymus T cells are first exposed to self protein antigens by the direct or indirect presentation of pTAs by mTECs (Mathis, D. et al., Nat Rev Immunol 7, 645-50 (2007); Gray, D. et al., J Exp Med 204, 2521-8 (2007); Anderson, M. S. et al., Science 298, 1395-401 (2002); Gavanescu, I. et al., Proc Natl Acad Sci USA 104, 4583-7 (2007); Gallegos, A. M. et al., J Exp Med 200, 1039-49 (2004); Klein, L. et al., Eur J Immunol 31, 2476-86 (2001)). Self-reactive T cells that have escaped central deletion can be further tolerized in the periphery by LNSCs (Lee, J. W. et al., Nat Immunol 8, 181-90 (2007); Magnusson, F. C. et al., Gastroenterology 134, 1028-37 (2008); Nichols, L. A. et al., J Immunol 179, 993-1003 (2007)). Our data shows that marrow-derived stromal cells naturally express these antigens in a similar fashion to mTECs and LNSCs and implicates the bone marrow as a potentially new site of pTA expression. The bone marrow is well known as a primary lymphoid organ that provides unique microenvironments that support lymphogenesis (Avecilla, S. T. et al., Nat Med 10, 64-71 (2004); Nagasawa, T., Nat Rev Immunol 6, 107-16 (2006)). But recent studies have shown that the marrow can also be considered as a secondary lymphoid organ which houses naïve, circulating B cells (Cariappa, A. et al., Immunity 23, 397-407 (2005)), long-lived plasma cells (Moser, K. et al., Curr Opin Immunol 18, 265-70 (2006); Tokoyoda, K. et al., Immunity 20, 707-18 (2004)), and mature CD4+, CD8+, and memory T cells (Cavanagh, L. L. et al., Nat Immunol 6, 1029-37 (2005); Mazo, I. B. et al., Immunity 22, 259-70 (2005); Masopust, D. et al., Science 291, 2413-7 (2001); Di Rosa, F. et al., Trends Immunol 26, 360-6 (2005)). These lymphocytes participate in distinct immune responses in situ. Bone marrow-resident B cells were shown to partake in T cell-independent humoral immune responses to blood-borne microbes by differentiating into antibody-secreting plasma cells (Cariappa, A. et al., Immunity 23, 397-407 (2005)). Resident T cells are thought to be primed to blood-borne pathogens as well as initiate full-blown memory responses from the bone marrow niche (Cavanagh, L. L. et al., Nat Immunol 6, 1029-37 (2005); Mazo, I. B. et al., Immunity 22, 259-70 (2005); Masopust, D. et al., Science 291, 2413-7 (2001); Di Rosa, F. et al., Trends Immunol 26, 360-6 (2005)). It is likely that in such a cellular milieu, a tolerance mechanism is required to inhibit self-reactivity. MSCs may be a part of such a mechanism in situ to directly or indirectly tolerize developing and/or mature lymphocytes to self antigens within the bone marrow. Furthermore, these lymphocytes are compartmentalized in perivascular spaces exactly where we had located UEA-1+ cells that are presumably MSCs. These spatial results are consistent with transplantation experiments with MSCs where we and others showed that transferred mouse (see Example 1) and human MSCs (Le Blanc, K. et al., Transplantation 79, 1607-14 (2005)) form clusters within engrafted tissues, the latter report focusing on the bone marrow. The close proximity with megakaryocytes may be relevant to physiological processes such MSC maintenance via megakaryocyte-mediated signals (e.g., TGF-β) or an MSC-specific effect on thrombopoiesis.

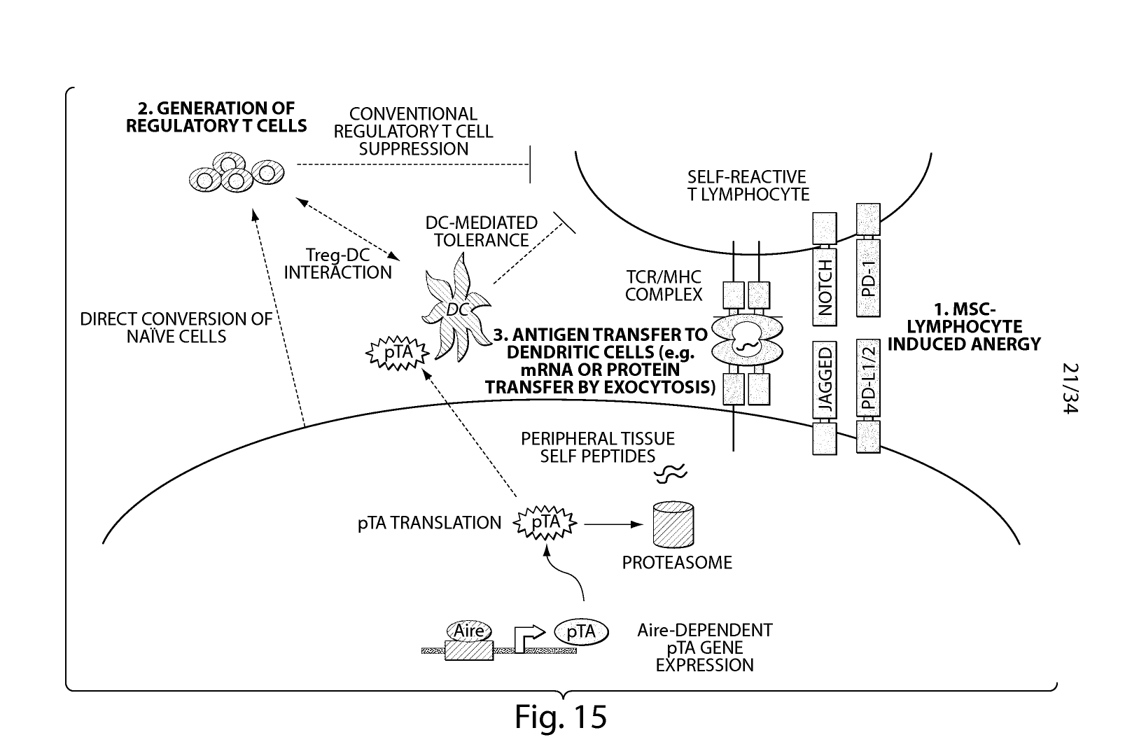

The expression of pTAs by MSCs may be causally related to the efficacy of these cells in various types of autoimmune disease (Augello, A. et al., Arthritis Rheum 56, 1175-86 (2007); Zappia, E. et al., Blood 106, 1755-61 (2005); Gerdoni, E. et al., Ann Neurol 61, 219-27 (2007); Lee, R. H. et al., Proc Natl Acad Sci USA 103, 17438-43 (2006)). We put forth an integrated theory of the therapeutic mechanism of action of MSC transplantation based on pTA expression in FIG. 15. Here, we depict our hypothesis of AIRE-dependent translation and presentation of pTAs. The first pathway involves direct contact between MSCs and lymphocytes, whereby the presentation of self-peptides along with negative costimulation leads to T cell anergy. In the second pathway, MSCs license DCs by serving as a reservoir of self antigens that are then phagocytosed by the dendritic cells to indirectly tolerize lymphocytes. The third pathway involves the direct generation of tolerizing DCs and/or the generation of regulatory T cells in situ. Ultimately, these pathways may all exist in concert to amplify the local immunosuppressive effects of the initial engrafted cell mass.

### Example 4

Mice. C57Bl/6 mice between 4 to 6 weeks of age were purchased from Charles River Laboratory. Ovalbumin (OVA)-specific (OT-I) T cell receptor transgenic mice and mice with a truncated, cytosolic form of OVA under the control of the intestinal fatty acid binding protein (iFABP) promoter (herein referred to as iFABP-tOVA) were maintained in the Dana Farber Cancer Institute's animal facility. The animals were cared for in accordance with the guidelines set forth by the Committee on Laboratory Resources, National Institutes of Health. All experimental procedures performed were approved by Subcommittee on Research Animal Care and Laboratory Animal Resources of Massachusetts General Hospital. Animals were maintained in a light-controlled room (12-h light-dark cycle) at an ambient temperature of 25° C. with chow diet and water ad libitum.

Isolation and Culture of Bone Marrow-Derived MSCs. Bone marrow was harvested from wild-type and iFABP-tOVA mice after euthanization. Tibias and femurs were dissected and the marrow space was flushed with MSC expansion medium using a 23 gauge needle. Bone marrow plugs were collected on ice, dissociated by repeated passage through an 18 gauge needle and passed through a 70 um filter to remove bony spicules and debris. Approximately 50×10⁶ bone marrow cells were plated on a 100 mm² tissue culture dish and cultured for 3 days to allow for differential adhesion of stromal cells. Nonadherent cells were aspirated on day 3 and the adherent population was cultured in MSC expansion medium for a subsequent 4-10 days to achieve the maximal number of colony forming unit-fibroblast prior to initial passage. Cells were passaged using 0.1% trypsin/0.1% EDTA, and subcultured at a density of 5×10³ cells/cm². All cultures were used between passages 2-8. Prior to use, stromal cells were then depleted of CD11b and CD45 cells using magnetic activated cell sorting (MACS) per vendor's instructions. Long term cultured MSCs were kindly donated by the Center for Gene Therapy at Tulane University and grown in MSC expansion medium. MSC expansion medium consisted of alpha-MEM without deoxyribonucleosides and ribonucleosides (Gibco), 10% lot selected FBS (Atlanta Biologicals), 100 U/ml penicillin (Sigma), and 100 μg/ml streptomycin (Sigma).

Total RNA Isolation and Endpoint/Quantitative RT-PCR. RNA was extracted from 0.1-1.0×10⁶ MSCs using the Nucleospin RNA purification kit (BD Biosciences, Palo Alto, Calif.) per the manufacturer's instructions. Approximately 500-1000 ng of total mRNA was reverse transcribed to cDNA using the Two-Step RT-PCR Kit (Qiagen, Valencia, Calif.) per manufacturer's instructions and amplified in a Perkin Etus Thermal Cycler 480 or a Stratagene Light Cycler. Cycling conditions for PCR were: 1) 50° C. for 30 minutes; 2) 95° C. for 15 minutes ; 3) 40 cycles at 94° C. for 30 seconds, 55° C. for 30 seconds, and 72° C. for 1 minute; and 4) a final extension step at 72° C. for 10 minutes. Primers used for amplification are listed in a previous Table. For quantitative analysis, we used a ΔCt method with stated controls with results stated as relative differences between experimental groups. For endpoint analysis, amplified cDNA was ran on a 1.2% agarose gel and visualized using a gel imager.

Transgenic Antigen Presentation Assay. OT-I T cells were isolated from the spleen and lymph nodes of OT-I mice. The cell suspensions were centrifuged at 1500 rpm for 10 min. and were exposed to ACK lysis buffer for 1-2 minutes to remove contaminating erythrocytes. ACK lysis buffer consisted of 8.024 mg NH₄Cl, 1.0 mg KHCO₃, 3.722 mg Na₂EDTA.2H₂O in a 1 liter solution of deionized H₂O adjusted to a pH of 7.4. The solution was neutralized with serum containing medium and pelleted. OT-I cell were incubated with 5 uM CFSE (Molecular Probes) for 10 min. at 37 degrees and subsequently depleted of CD4, CD19, and CD11b cells using MACS to enhance the purity of these cells. Labeled OT-I cells were cocultured with antigen presenting cells (CD45+ stromal cells, CD45− MSCs, or bone marrow-derived dendritic cells) with or without prior incubation with OVA. Proliferation of lymphocytes was measured after 60 hours by dilution of fluorescent label upon cell division by flow cytometry.

Coculture of Splenocytes and MSCs. Spleens of C57Bl/6 mice (aged 12 weeks) were dissected from healthy mice and dissociated into cellular components by mechanical disruption of the tissue into a saline solution. The cell suspensions were centrifuged at 1500 rpm for 10 minutes and were exposed to ACK lysis buffer for 1-2 minutes to remove contaminating erythrocytes. The solution was neutralized with serum containing medium and pelleted. Splenocytes were fractionated using CD11b or CD25 microbeads (Miltenyi Biotec, Auburn, Calif.) per vendor's protocols. Whole or fractionated splenocytes (1×10⁶ cells) were cultured alone or in coculture with MSCs at a 1:10 ratio of splenocyte:MSC in RPMI medium with 10% FCS, low-dose recombinant human IL-2 (10 U/ml; R&D Systems, Minneapolis, Minn.), 100 U/ml penicillin and 100 μg/ml streptomycin. After 5 days of coculture, splenocytes were analyzed for expression of CD4, CD25, and Foxp3 by flow cytometry.

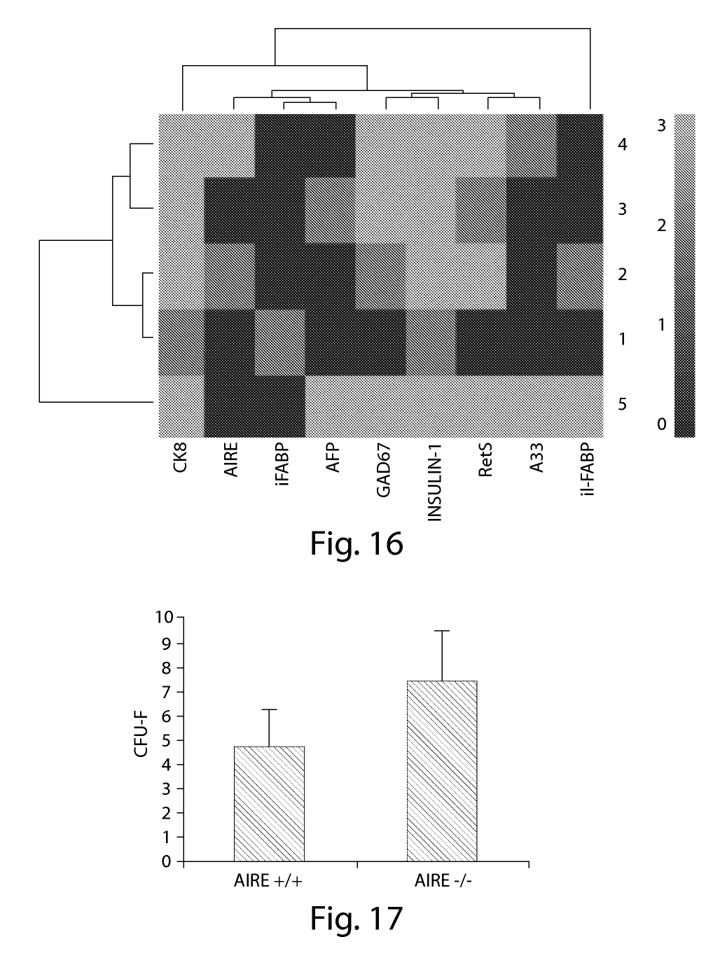

Effects of AIRE on MSC Function. We sought to determine whether expression of particular pTAs varies among different colonies (clones) of BMSCs. Five colonies of BMSCs were isolated, subcultured and passed three times as described above. In FIG. 16, relative expression level, as detected by RT-PCR, of various markers (indicated on the x-axis) in the five clones (indicated on the y-axis) are shown. Markers that were not detected in a particular clone are indicated by a black box, whereas markers that were highly expressed are indicated by a bright red box. Moderate marker expression is indicated by darker red boxes. For example, AFP expression was not detected in clones 1, 2 or 4, but AFP was highly expressed in clone 5 and was moderately expressed in clone 3.

Based on the results shown in FIG. 16, it was concluded that expression of pTAs is variable from cell-to-cell (i.e., colony-to-colony) in bone marrow stroma cells prior to their in vitro colonization and therefore may be amenable to creation of tailored cell clones and banks.

We also sought to determine whether AIRE affects BMSC viability. BMSCs were isolated and cultured for 10 days as previously described. As shown in FIG. 17, significantly fewer wild-type (AIRE+) colonies than AIRE-deficient colonies were present in separate cultures grown under identical conditions. These data suggest that AIRE negatively affects BSMC viability.

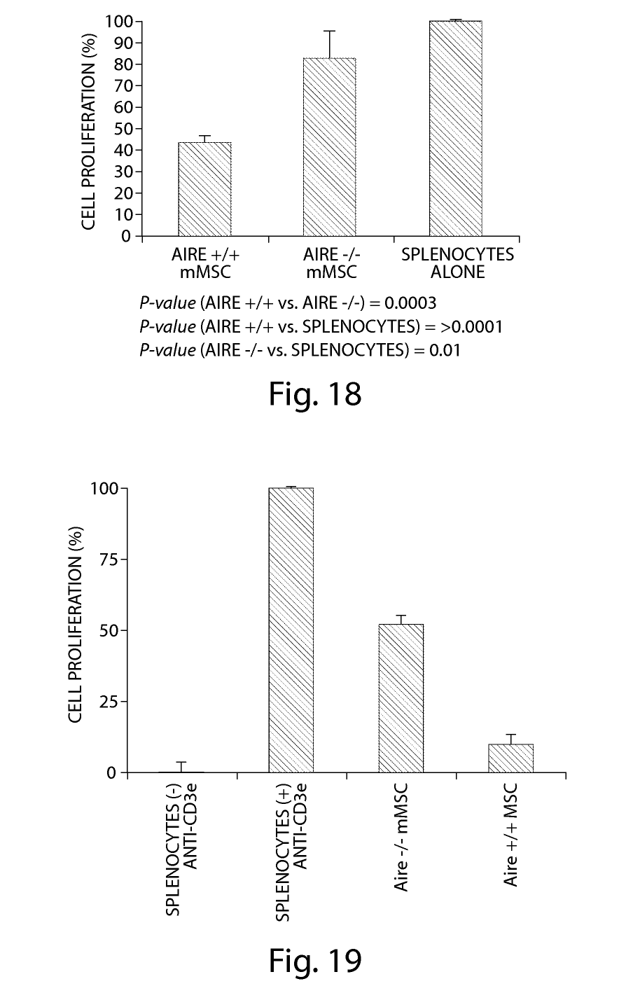

We further sought to compare the proliferation of AIRE-deficient murine CD45− BMSCs co-cultured with splenocytes in the presence of anti-CD3e with the proliferation of wild-type murine CD45− BMSCs grown under the same conditions. Such comparison was carried out in two separate experiments, the results of which are shown in FIGS. 18 and 19.

Based on the results shown in FIGS. 18 and 19, it was determined that AIRE-deficient BMSCs exhibit a multiple-fold loss of T cell suppression capability as compared to wild-type cells.

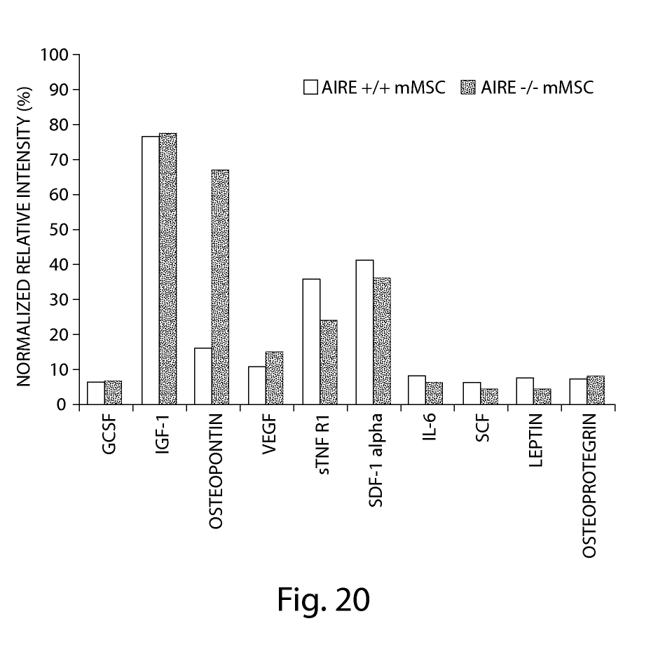

We also sought to examine expression of various markers in bone marrow stroma cells from wild-type and AIRE-deficient mice. Bone marrow stroma cell lysates were obtained from CD45− wild-type and AIRE-deficient mice 10 days after cell isolation. As shown in FIG. 20, the relative protein level of various markers (shown along the x-axis) was determined in the CD45− wild-type and AIRE-deficient lysates. While no significant difference was observed in the level of any one of GCSF, IGF-1, VEGF, sTNF R1, SDF-1 alpha, IL-6, leptin or osteoprotegrin when wild-type lysate was compared to AIRE-deficient lysate, the AIRE-deficient lysate contained more than four times more osteopontin than that amount of osteopontin that was present in the wild-type lysate.

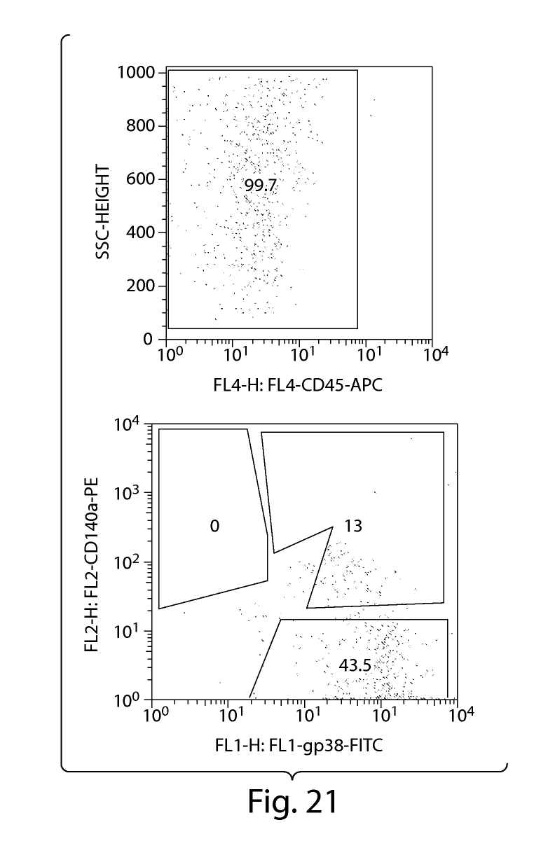

Based on these data, it was concluded that osteopontin is expressed at a significantly higher level in AWE-deficient bone marrow stroma cells than it is in wild-type bone marrow stroma cells, thus showing that AIRE affects the secreted proteins of stromal cells. We further sought to determine whether CD45− MSCs express PDGF-β and/or gp38. Murine MSCs were purified after initial isolation and subcultured for two passages as described above. As shown in FIG. 21, dotplot data obtained indicated that both PDGF-β and gp38 are expressed in MSCs.

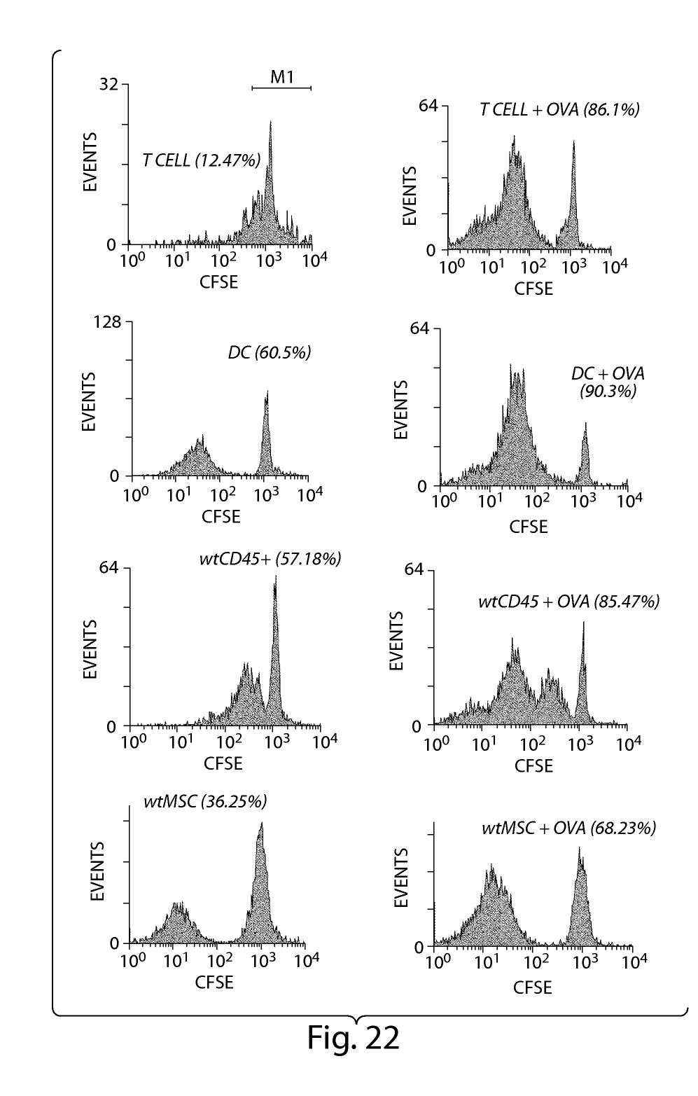

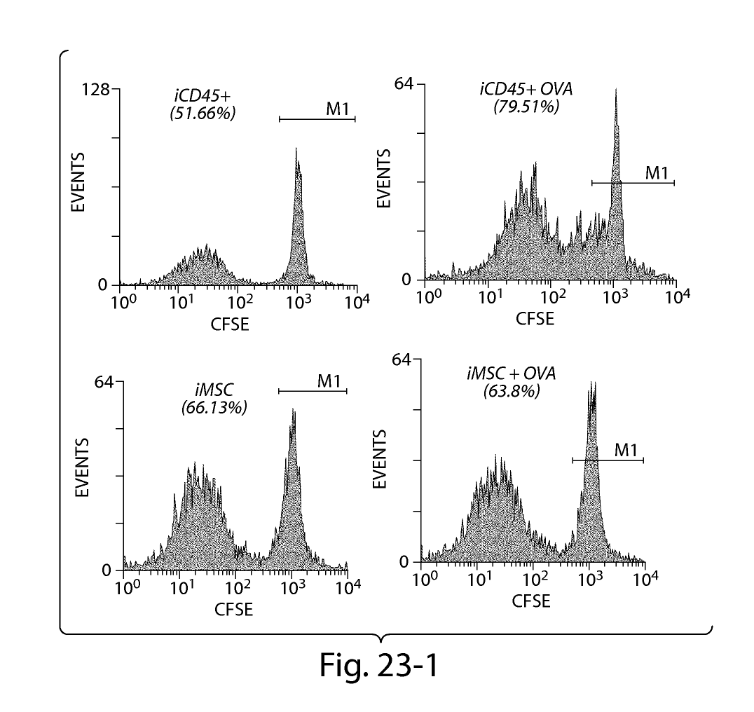

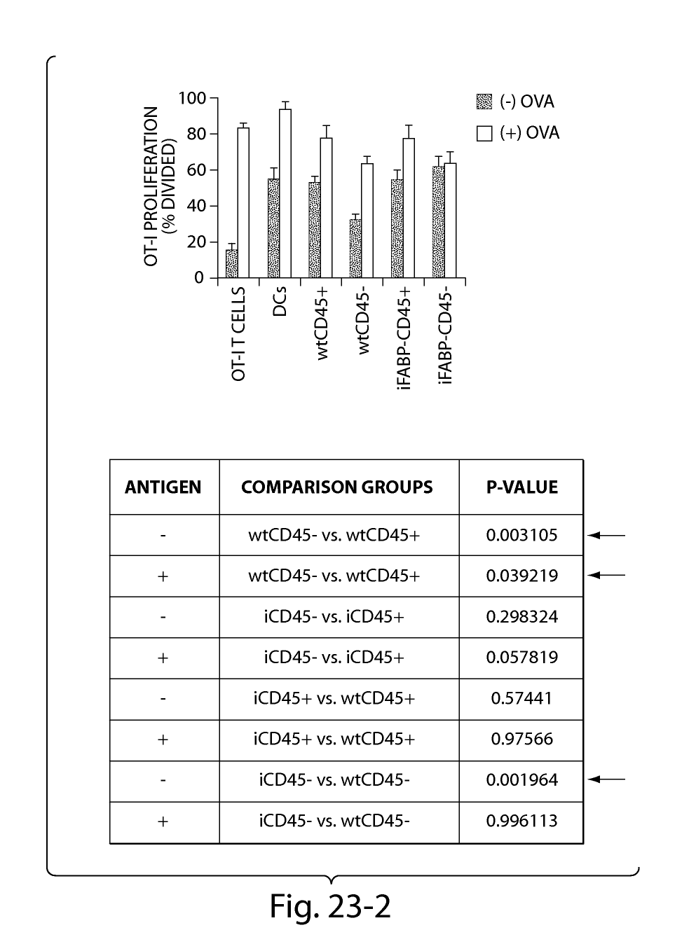

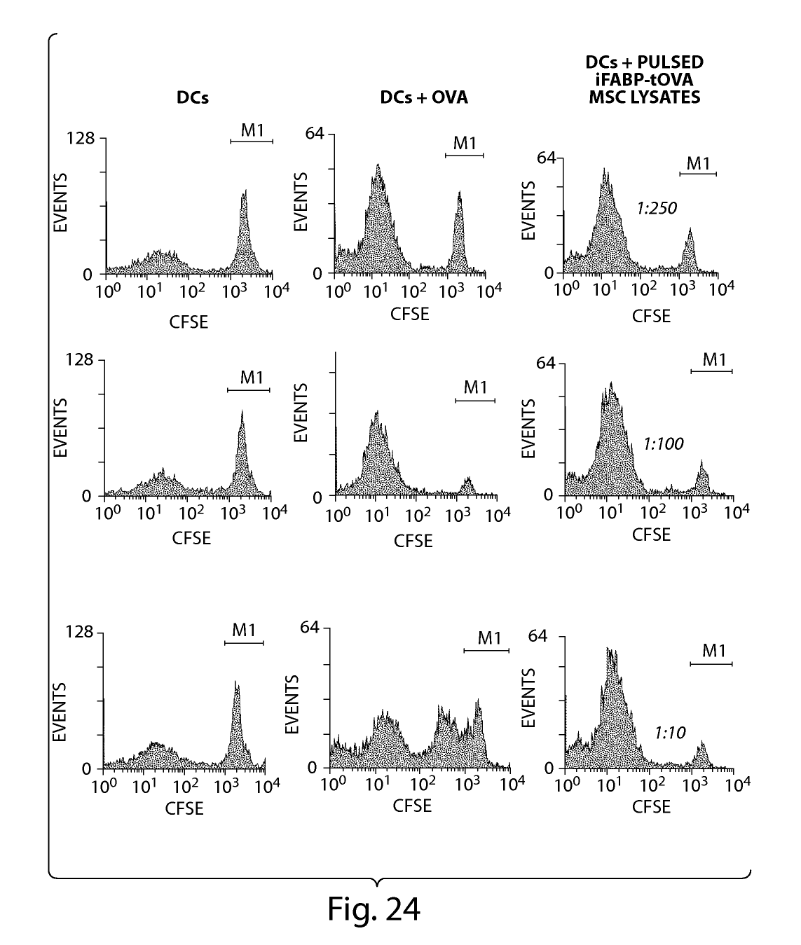

Functional Antigen Presentation and Antigen Transfer by Transgeneic MSCs. We further sought to determine whether CD45− MSCs can functionally present pTAs to antigen-specific T lymphocytes. The experiments consisted of (a) T lymphocytes specific for a target ovalbumin (OVA) antigen, and (b) a cocultured antigen presenting cell (dendritic cell, CD45+ marrow stromal cell, or CD45− marrow stromal cell) isolated from wild-type (wt) mice or mice genetically engineered to express OVA driven by a pTA promoter, iFABP (termed iFABP-tOVA). Panels FIGS. 22 and 23 collectively show that wild-type CD45− MSCs cannot cause the stimulation of OVA T cells, without being primed with OVA antigen to present. On the contrary, CD45− MSCs from iFABP-tOVA mice cause T cell proliferation independent of being pulsed with OVA antigen. These results demonstrate that MSCs can present an antigen that is driven by a pTA promoter to antigen-specific cells. FIG. 24 demonstrates that the transfer of MSC intracellular components from iFABP-tOVA CD45− MSCs can stimulate OVA-specific T cells. These data demonstrate that the transfer of species including proteins (e.g. OVA protein), lipids (including microvesicles containing OVA protein, and/or OVA mRNA), carbohydrates, organelles, RNA (e.g. mRNA for OVA), DNA can be incorporated into another cell (e.g., an antigen presenting dendritic cell) to lead to a functional response.

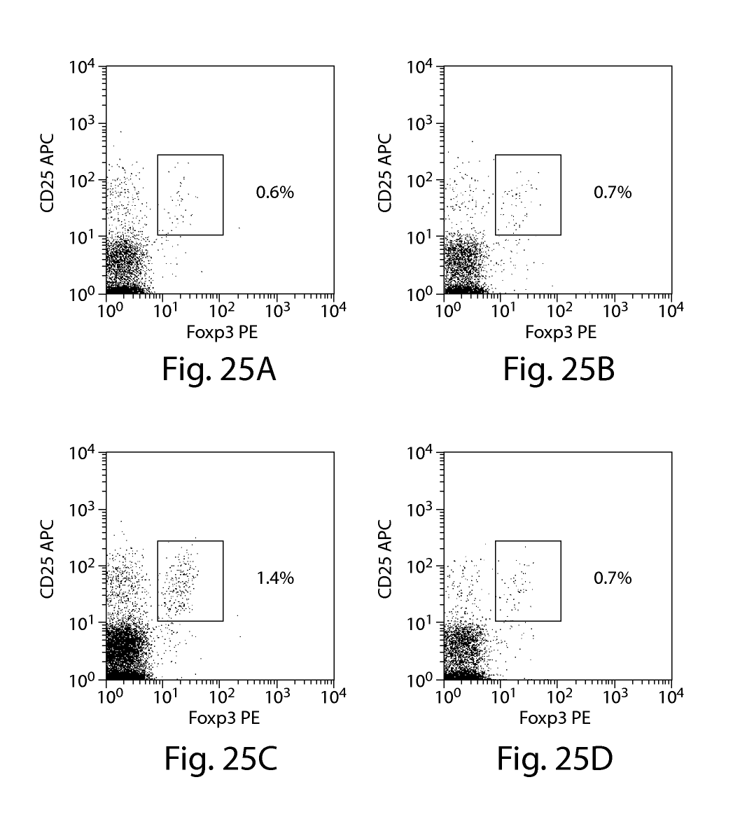

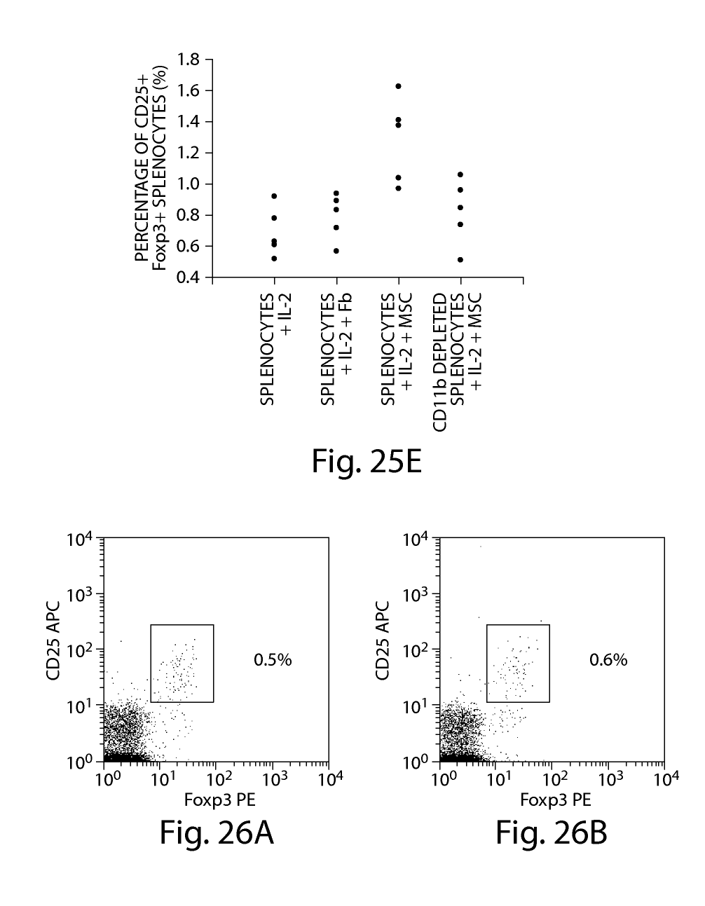

Derivation of Therapeutic CD11b+ Cells by MSC Interaction and Antigen Transfer. We sought to identify a therapeutic consequence of antigen transfer. These experiments consist of coculturing CD45− MSCs with splenocyte populations in culture conditions that favor the formation of CD25+ Foxp3+ suppressor T cells. FIG. 25. demonstrates that direct coculture of CD45− MSCs with whole splenocytes in IL-2 supplemented medium leads to an increase in suppressor T cells compared to a no cell or mock fibroblast cell control. The generation of suppressor cells by MSCs was lost if CD11b+ cells were not present. These data suggest that MSCs interact with an intermediate cell type to increase suppressor T cell number.

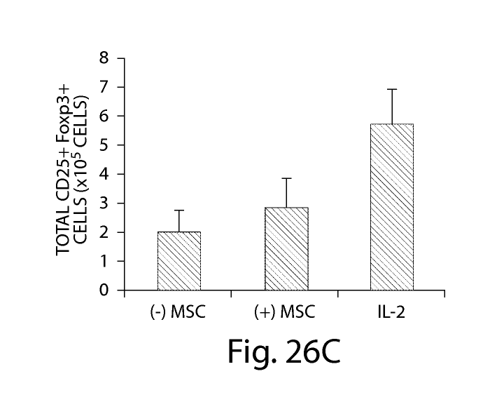

FIG. 26. shows that MSCs in coculture with CD25− or CD25+ splenocytes does not increase the number of suppressor cells. These studies show that MSCs do not directly increase this population of suppressor cells, but act indirectly.

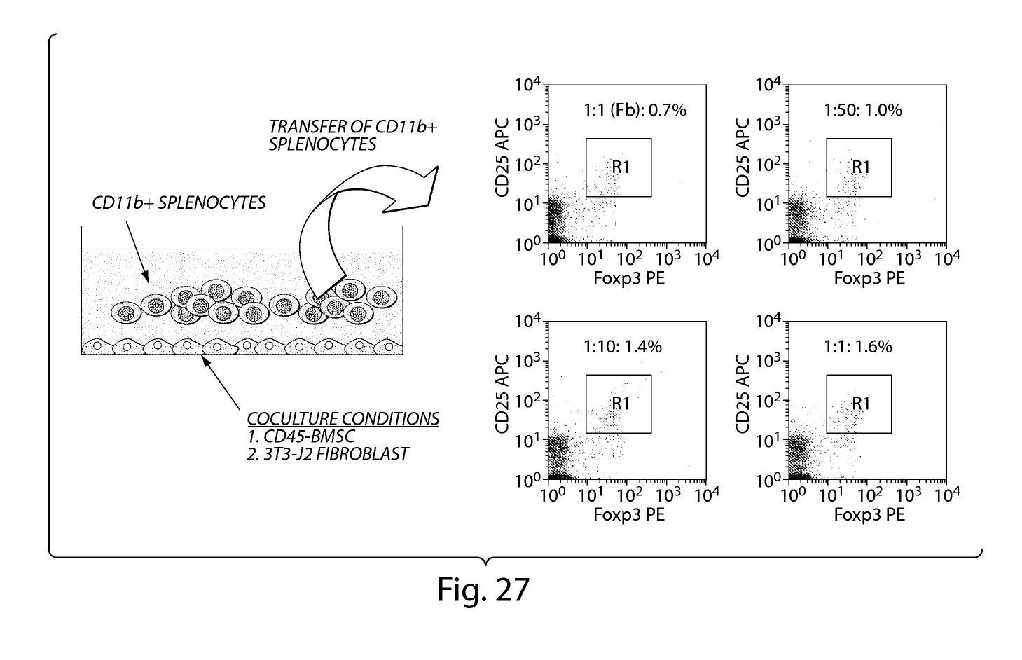

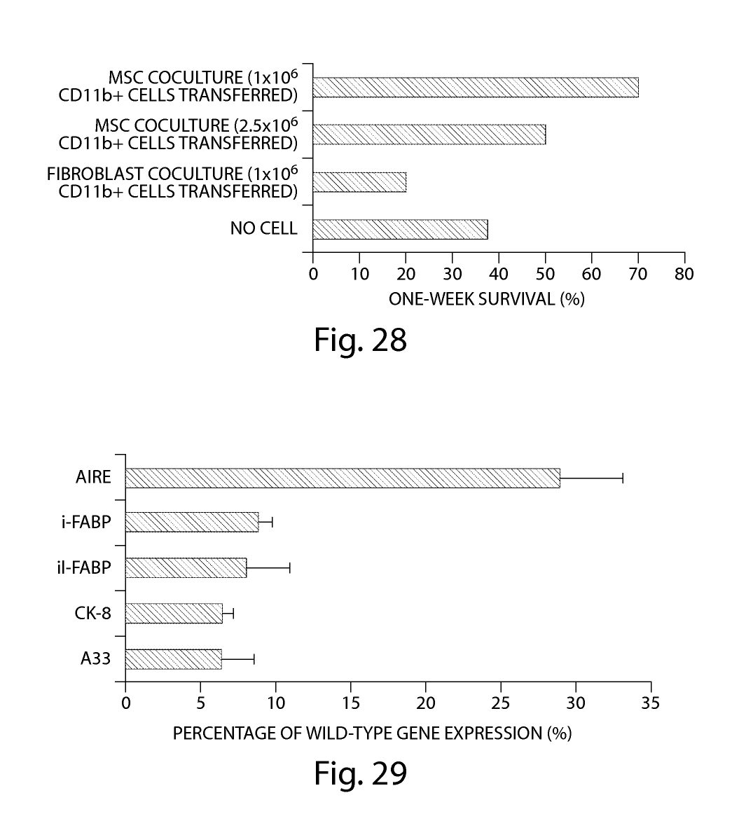

We sought to test whether CD11b+ cells, that have interacted with MSCs, could be transferred into a whole splenocyte culture and cause the increase in Foxp3+ cells. FIG. 27 shows a dose-dependent increase in the number of suppressor T cells as a function of the number of CD11b+ cells that had been previously cocultured with MSCs. Fibroblast coculture did not lead to an increase in suppressor cells. These studies demonstrate that MSCs can reprogram other antigen presenting cells to cause a therapeutic response. Indeed, FIG. 28 shows that the transfer of MSC cocultured CD11b+ cells into colitic mice leads to a survival benefit in these animals demonstrating the in vivo therapeutic relevance of antigen transfer.

We sought to determine if pTA expression is affected by chemical stimuli. FIG. 29 shows that the levels of pTA and AIRE decrease upon exposure to IFN-gamma. These data show that the levels of pTAs can be controlled by exogenous stimuli.

### Equivalents

It should be understood that the preceding is merely a detailed description of certain embodiments. It therefore should be apparent to those of ordinary skill in the art that various modifications and equivalents can be made without departing from the spirit and scope of the invention, and with no more than routine experimentation.

All references, patents and patent applications that are recited in this application are incorporated by reference herein in their entirety.

What is claimed is:

1. A method for preparing an isolated mesenchymal stem cell population having a defined antigen expression profile comprising determining an antigen expression profile in an isolated mesenchymal stem cell population, and
physically separating the isolated mesenchymal stem cell population based on antigen expression to generate one or more isolated mesenchymal stem cell populations having defined antigen expression profile.

2. The method of claim 1, wherein the antigen expression profile is a peripheral tissue antigen expression profile.

3. The method of claim 1, wherein the antigen expression profile is an antigen expression profile for a single antigen.

4. The method of claim 1, wherein the antigen expression profile is an antigen expression profile for multiple antigens.

5. The method of claim 1, wherein the isolated mesenchymal stem cell population is physically separated based on type of antigen expression.

6. The method of claim 1, wherein the isolated mesenchymal stem cell population is physically separated based on type and level of antigen expression.

7. The method of claim 1, wherein the isolated mesenchymal stem cell population is a bone marrow mesenchymal stem cell population.

8. A method for preparing a mesenchymal stem cell having a defined antigen profile comprising
expressing an exogenous nucleic acid comprising a peripheral tissue antigen in a mesenchymal stem cell.

9. (canceled)

10. An isolated mesenchymal stem cell population having a defined antigen expression profile.

11. An isolated mesenchymal stem cell population generated according to claim 1.

12. A mesenchymal stem cell bank comprising
a plurality of isolated mesenchymal stem cell populations of claim 10.

13. (canceled)

14. A method for treating a subject having or at risk of developing an autoimmune disease comprising
administering to a subject in need thereof an isolated mesenchymal stem cell population having a defined antigen expression profile in an effective amount to treat the subject.

15. A method for treating a subject having or at risk of developing an autoimmune disease comprising
administering to a subject in need thereof an isolated mesenchymal stem cell population prepared according to the method of claim 1 in an effective amount to treat the subject.

16-17. (canceled)

18. A method for modulating an immune response comprising
administering to a subject in need thereof an isolated mesenchymal stem cell population having a defined antigen expression profile in an effective amount to treat the subject.

19. A method for modulating an immune response comprising
administering to a subject in need thereof an isolated mesenchymal stem cell population prepared according to the method of claim 1 in an effective amount to modulate an immune response.

20-22. (canceled)

23. A method for treating a subject having or at risk of developing an autoimmune disease comprising
administering to a subject in need thereof a mesenchymal stem cell lysate lipophilic fraction in an effective amount to treat the subject.

24. A method for treating a subject having or at risk of developing an autoimmune disease comprising
administering to a subject in need thereof a mesenchymal stem cell conditioned media lipophilic fraction in an effective amount to treat the subject.

25-26. (canceled)

27. A method for preparing an MSC antigen presenting cell comprising
in vitro contacting a naïve antigen presenting cell with a mesenchymal stem cell lysate or conditioned media, and
allowing sufficient time for the naive antigen presenting cell to express on its surface an antigen or fragment thereof from the mesenchymal stem cell lysate or conditioned media, thereby generating an MSC antigen presenting cell.

28-38. (canceled)

39. A method for modulating an immune response comprising
administering to a subject in need thereof an MSC antigen presenting cell prepared according to the method of claim 27 in an effective amount to modulate an immune response.

40-41. (canceled)

42. A method for identifying a candidate mesenchymal stem cell comprising
contacting a mesenchymal stem cell with an antigen-specific activated immune cell, and
measuring antigen-specific activity of the antigen-specific activated immune cell prior to and after contact with the mesenchymal stem cell,
wherein a reduction in antigen-specific activity as a result of contact with the mesenchymal stem cell identifies a candidate mesenchymal stem cell.

43-44. (canceled)
